(19) United States
(12) Patent Application Publication
(10) Pub. No.: US 2018/0327768 A1
(43) Pub. Date: Nov. 15, 2018

(54) TARGETED DELIVERY OF THERAPEUTIC PROTEINS BIOENCAPSULATED IN PLANT CELLS TO CELL TYPES OF INTEREST FOR THE TREATMENT OF DISEASE

(71) Applicant: THE TRUSTEES OF THE UNIVERSITY OF PENNSYLVANIA, Philadelphia, PA (US)

(72) Inventor: Henry Daniell, Media, PA (US)

(21) Appl. No.: 15/776,602

(22) PCT Filed: Nov. 16, 2016
(86) PCT No.: PCT/US2016/062371

§ 371 Date: May 16, 2018

Related U.S. Application Data
(60) Provisional application No. 62/256,053, filed on Nov. 16, 2015.

(51) Int. Cl. C12N 15/82; C07K 14/435; C07K 14/705; C07K 14/47; C07K 14/28; A61K 36/00; A61K 9/00; A61K 9/48

(52) U.S. Cl. CPC C12N 15/8257; C07K 14/435; C07K 14/705; C07K 14/4723; A61K 38/00; A61K 36/00; A61K 9/0053; A61K 9/48; C07K 14/28

(57) ABSTRACT

Compositions and methods for producing therapeutic fusion proteins comprising targeting sequences directing the protein to cells or tissues of interest are disclosed.

This application claims priority to U.S. Provisional Application No. 62/256,053 filed Nov. 16, 2015, the entire disclosure being incorporated herein by reference as though set forth in full.

The United States Government has rights in this invention which was made with funds from the National Institutes of Health, Grant Numbers: R01 GM 63879, R01 HL 109442, and R01 HL 107904.

### FIELD OF THE INVENTION

This invention relates to the fields of transplastomic plants and low cost protein drug production and delivery. More specifically, the invention provides plants comprising chloroplast expressed transgenes encoding therapeutic proteins and peptides operably linked to targeting sequences directing the plant cell encapsulated fusion proteins to tissues of interest upon processing in the gut.

### BACKGROUND OF THE INVENTION

Several publications and patent documents are cited throughout the specification in order to describe the state of the art to which this invention pertains. Each of these citations is incorporated herein by reference as though set forth in full.

Biopharmaceuticals produced in current systems are prohibitively expensive and are not affordable for a large majority of the global population. In the US, the average annual cost of protein drugs is 25-fold higher than for small molecule drugs. The cost of protein drugs ($140 billion in 2013) exceeds the GDP of >75% of countries around the globe, making them unaffordable in these countries [1]. One third of the global population earning <$2 per day cannot afford any protein drugs. Although recombinant insulin has been sold commercially for five decades, it is still not affordable for a large majority of global population. This is because of production of such drugs is prohibitively expensive, often requiring costly fermenters, multi-step purification procedures, cold storage/transportation, means for sterile delivery. Additionally, the short shelf life of these drugs is associated with increased cost. Oral delivery of protein drugs has been elusive for decades because of their degradation in the digestive system and inability to cross the gut epithelium for delivery to target cells.

However, several recent studies have indirectly shown that plant cell wall protects expressed protein drugs from acids and enzymes in the stomach via bio-encapsulation [2, 3]. Human digestive enzymes are incapable of breaking down glycosidic bonds in carbohydrates that make up plant cell wall. However, when intact plant cells containing protein drugs reach the gut, commensal microbes could digest plant cell wall and release protein drugs in the gut lumen. Bacteria inhabiting the human gut have evolved to utilize complex carbohydrates in plant cell wall and are capable of utilizing almost all plant glycans [4, 5]. Fusion of the (nontoxic) Cholera toxin B subunit (CTB) to green fluorescent protein (GFP) expressed in chloroplasts and bioencapsulated in plant cells was delivered across the gut epithelium through GM1 receptors and GFP was released into the circulatory system [6]. Fusion of CTB to therapeutic proteins facilitates their effective oral delivery for induction of oral tolerance [7-11] or functional proteins to sera [12-14] or even across blood brain or retinal barriers [15, 16].

Foreign proteins can also be delivered into living cells by fusion with protein transduction domains (PTDs) with cell membrane penetration properties that do not require specific receptors [17]. The peptide and protein transduction domains (PTDs) are small cationic peptides containing 8-16 amino acids, and most frequently function as transporter for delivery of macromolecules [17]. PTDs carry molecules into cells by a receptor independent, fluid-phase macro-pinocytosis, which is a special form of endocytosis. Although different PTDs show similar characteristics of cellular uptake, they vary in their efficacy for transporting protein molecules into cells. The efficacy for cellular uptake has been found to correlate strongly with the number of basic amino acid residues. Since PTDs have been shown to deliver biologically active proteins in cultured mammalian cells and in animal model in vivo and in vitro [18-20], the PTD fused protein delivery method should have great therapeutic drug delivery potential. T and B lymphocytes are major cellular components of the adaptive immune response, but their activation and homeostasis are controlled by dendritic cells. B cells can recognize native Ag directly through B cell receptor on their surface and secrete antibodies. However, T cells are only able to recognize peptides that are displayed by MHC class I and II molecules on the surface of APCs. Macrophage is one type of professional antigen-presenting cells, having many important roles including removal of dead cells and cell debris in chronic inflammation and initiating an immune response [21,22]. Macrophages participate in the orchestration of primary and secondary immune responses. Mast cells are involved in generating the first inflammatory response during infection, which is important for initiating innate and adaptive immunity. When activated, a mast cell rapidly releases its characteristic granules and various hormonal mediators into the interstitium. Therefore, mast cells play important roles in wound healing, allergic disease, anaphylaxis and autoimmunity.

Dendritic cells are important immune modulatory cells. Dendritic cells form a complex with multifunctional APCs and play critical roles in anti-pathogen activities. Moreover, dendritic cells differentiate into different types of functional cells, stimulated by different antigens and induce humoral or cellular immunity. Conversely, DCs are also critical for the homeostasis of regulatory T cells (Treg), extrathymic induction of Treg, and for immune tolerance induction in transplantation and treatment of allergy or autoimmune disease. The tissue microenvironment, activation signals, and subsets of DCs are important parameters that determine whether antigen presentation by DCs result in immunity or tolerance [23-25]. Therefore, targeted in vivo delivery of antigens to DCs may not only be useful for inducing tumor-specific immune responses and establishing novel strategies for vaccine development, cancer immunotherapy, but also for tolerance induction protocols [26-28]. For example, the gut associated lymphatic tissues (GALT) provide the largest surface area for antigen entry into the body and a very unique microenvironment with tolerogenic properties, including expression of the immune suppressive cytokines IL-10 and TGF-β [29-31]. Gut epithelial cells and CX1CR5⁺ macrophages sample antigens from the gut lumen. In particular in the endothelium of Peyer's patches, microfold cells (M cells) endocytose and phagocytose antigens to channel these to DCs. CD11c⁺ DCs in the gut contain a high proportion of CD103⁺ DC, which express TGF-β preferentially induce Treg [32]. Recently, we demonstrated that oral tolerance induction to coagulation factors in hemophilic mice upon delivery of bioencapsulated CTB-fusion antigens was associated with increased CD103⁺ DC frequency, antigen uptake by CD103⁺ DC, and induction of several subsets of Treg [9,10]. Also increased were plasmacytoid DC, which also have important immune modulatory functions [33]. DC peptide (DCpep) has been developed as a ligand to mucosal DCs [26]. This small peptide binds to a DC-specific receptor, and facilitates transportation of macromolecules into DC.

### SUMMARY OF THE INVENTION

In accordance with the present invention, a method for targeted delivery of therapeutic proteins to tissues or cells of interest in a subject in need thereof is disclosed. An exemplary method comprises administering an effective amount of plant cells or remnants thereof comprising a plastid expressed nucleic acid encoding said therapeutic protein operably linked to a fusion peptide sequence, expression of said nucleic acid causing production of a therapeutic fusion protein that selectively penetrates target cells or tissue of interest in vivo upon ingestion or administration of said plant or remnant thereof, thereby selectively delivering said therapeutic protein to targeted cells or tissues of said subject. Preferably, plants are green leafy vegetables, including, without limitation, lettuce, low nicotine tobacco, spinach, cabbage, and kale. Other plants include eggplant, carrot and tomato. In one embodiment, the fusion peptide sequence is a PTD. In another embodiment the fusion peptide sequence is a DC peptide. The fusion peptide can also be an antimicrobial peptide such as PG1 or RC101. In certain embodiments, the fusion peptide is a CTB peptide. In other embodiments of the invention, use of CTB fusion peptide is excluded, particularly when targeting needs to be more specific. Cells to be targeted include, without limitation, immune cells, somatic cells and dendritic cells. Tables 2, 3 and 4 provide therapeutic molecules useful in the plastid produced fusion proteins of the invention.

In one aspect, the therapeutic fusion protein is for the treatment of an endocrine disorder and the protein is selected from the group consisting of insulin, somatotropin, and insulin-like growth factors. The therapeutic fusion protein can also be used for the maintenance of hemostasis or prevention of thrombosis. In this embodiment, the therapeutic fusion protein is selected from the group consisting of a blood clotting factor, anti thrombin, protein C and tissue plasminogen activator. The method of the invention can also be used for treatment of subjects having an enzyme deficiency and the therapeutic fusion protein includes a protein selected from the group consisting of beta gluco-cerebrosidases, alpha-glucosidases, lactase, lipase, amylase, protease, and proteinase inhibitor.

The therapeutic fusion protein may augment existing biological pathways and is selected from the group consisting of erythropoietin, granulocyte colony stimulating factor, granulocyte macrophage colony stimulating factor, follicle stimulating hormone, chorionic gonadotropin, alpha interferon, interferon B, PDGF, Keratinocyte growth factor and bone morphogenic protein.

Another aspect of the invention includes a plant or plant cell comprising the therapeutic fusion proteins described above. The plants may be freeze dried. They may be in powdered from and optionally encapsulated. In a preferred embodiment, the therapeutic fusion protein is stable for months at ambient temperature.

### BRIEF DESCRIPTION OF THE DRAWINGS

FIGS. 1A-1F. Schematic diagrams for predicted protein structures and characterization of transplastomic lines expressing GFP-fusion proteins. (FIG. 1A) Interaction of CTB fusion protein and GM1 receptor, and predicted 3D structure of both PTD and DCpep. Pentasaccharide moiety of GM1 receptor establishes interaction with pentameric structure of CTB. The Hinge sequence for avoiding steric hindrance and furin cleavage site for releasing the tethered protein were placed between CTB and the fused protein. Computational predicted three-dimensional structures of both PTD and DCpep were obtained from iterative threading assembly refinement (I-TASSER) server [40]. The structure is shown in rainbow, where the color changes from blue to red gradually for residues from N-terminal to C-terminal (blue-green-yellow-orange-red). Among predicted structures, the model with the highest reliable structure for each peptide, which was chosen based on the combined results from parameter calculations such as confidence score (C-score), high-resolution models with root mean square deviation (RMSD) value, and template modeling score (TM-score), was presented. (FIG. 1B) Schematic diagram for expression cassette of GFP-fused carrier proteins and flanking regions. Prrn, rRNA operon promoter; aadA, aminoglycoside 3′-adenylytransferase gene; Ppsb A, promoter and 5′ UTR of psb A gene; CTB, coding sequence of non-toxic cholera B subunit; PTD, coding sequence of protein transduction domain; DCpep, dendritic cell binding peptide sequence; smGFP, gene sequence for soluble-modified green fluorescent protein; TpsbA, 3′ UTR of psb A gene; trnI, isoleucyl-tRNA; trnA, alanyl-tRNA. Restriction enzymes used for Southern blot analysis were indicated as BamHI/BglII for the generation of probe and HindIII for the digestion of genomic DNA. (FIG. 1C) Southern blot analysis of each transplastomic line expressing GFP-fused tag proteins. HindIII-digested gDNAs were probed with the flanking region fragment described above. (FIG. 1D) GFP fluorescence signals from each transplastomic line were confirmed under UV light. The picture was taken after 2 months of germination. Bar represents 0.5 cm. (FIG. 1E) Western blot analysis for densitometric quantification with GFP standard proteins. Lyophilized (10 mg) and fresh leaf material (100 mg) were extracted in 300 μL extraction buffer. 1× represents 1 μL of homogenate resuspended in the extraction buffer in a ratio of 100 mg to 300 μL. (FIG. 1F) Amount of GFP fusion proteins in fresh (F) and lyophilized (L) leaves. Data are means±SD of three independent experiments.

FIGS. 2A-2B. Efficiency of oral delivery and biodistribution of GFP fused with different tags. Serum (FIG. 2A) and tissue (FIG. 2B) GFP levels in mice (N=6 per group) fed leaf materials expressing CTB-GFP, PTD-GFP and DCpep-GFP. Adult mice were orally fed with leaf materials from transgenic tobacco plants, with the amount adjusted to GFP expression levels, for three consecutive days. A control group (N=6) kept unfed. Blood samples were collected at 2 and 5 hours after last gavage at which, mice were sacrificed and tissue samples were collected for protein isolation. GFP concentration in serum and tissues were measured with ELISA. The data was shown as average±SEM. Statistic significance was determined by a paired Student's t test, and p value less than 0.05 were considered significant. * P<0.05, ** P<0.01, *** P<0.05 or P<0.01 (CTD, PTD and DCpep versus Naïve)

FIGS. 3A-3L. Visualization of GFP in cells of ileum and liver of mice after oral delivery of plant cells. GFP delivery to small intestine (left panel). Shown are cross-sections stained with anti-GFP (green signal; Alexa Fluor 488), UEA-1 (which stains, among other cells, M cells, red signal, rhodamine), and DAPI (nuclear stain, blue). (FIGS. 3A-C). PTD-GFP delivery. (FIG. 3B) No primary antibody (NC: negative control). (FIGS. 3D-E) CTB-GFP delivery. (FIG. 3F) DCpep delivery. Original magnification: 200× (FIGS. 3A, B, D-F, insert in C) or 40× (FIG. 3C). GFP stain shown in liver's cryosection (right panel), identical exposure time during image capture. The primary antibody: rabbit anti-GFP antibody at 1:1000 and second antibody: Alexa Fluor 488 Donkey Anti-Rabbit IgG was used for GFP staining. (G, I and K) liver sections of mice fed with untransformed lyophilized plant cells. (FIGS. 3H, J and L) GFP signals of liver sections from mice fed with lyophilized plant cells expressing DCpep-GFP (FIG. 3H), PTD-GFP (FIG. 3J), and CTB-GFP (FIG. 3L). Original magnification: 100×.

FIGS. 4A-4F. Characterization of purified GFP fused proteins. (FIG. 4A) Quantification of purified GFP fused proteins, and coomassie staining and fluorescence image. Densitometric assay with western blot image was done with known amount of GFP standard protein to quantify the purified tag-fused GFP proteins. Purified proteins were run on SDS-PAGE and immunoprobed with anti-GFP antibody. Loading amounts were indicated as shown. Purity was calculated as a percentage of the amount detected on the immunoblot assay to total loading amount. (FIG. 4B) Coomassie staining of purified GFP tagged proteins. M, protein molecular weight marker; lane 1, PTD-GFP (10 μL, 2.37 μg); lane 2, Dcpep-GFP (40 μL, 3.12 μg), lane 3, CTB-GFP (10 μL, 32.8 μg), and lane 4, GFP (400 ng). (FIG. 4C) Non-denaturing SDS-PAGE of purified GFP fusion proteins in order to determine GFP fluorescence. Lane 1 (PTD-GFP 10 μl, 9.17 μg TSP loading), lane 2 (DCpep-GFP 15 μl, 4.6 μg TSP loading) and lane 3 (CTB-GFP 20 μl, 33 μg TSP loading). (FIGS. 4D and 4E). The purified GFP-tagged proteins were examined for their binding affinity to GM1 receptor. Anti-CTB (FIG. 4D) and anti-GFP (FIG. 4E) antibody were used to detect the interaction between GM1 and the GFP fusion proteins. The protein amounts used for the assay are as follows. CTB, 10 pg; CTB-GFP, 1.25 ng; PTD-GFP 10 ng; DCpep-GFP 10 ng; GFP, 10 ng and UT, untransformed wild type total proteins, 100 ng. (FIG. 4F). Non-denaturing Tris-tricine PAGE of purified CTB-GFP to determine pentameric structure. Pentameric structure of purified CTB-GFP was immunoprobed using anti-CTB antibody (1 in 10,000). Loading amounts of CTB-GFP are indicated as in the figure.

FIGS. 5A-5B. Uptake of GFP fused with different tags by human immune and non-immune cells (FIG. 5A) Translocation of purified GFP fusion proteins in human cell lines. 2×10⁴ cells of cultured human dendritic cell (DC), B cell, T cell and mast cells were incubated with purified GFP fusion: CTB-GFP (8.8 μg/100 μl PBS), PTD-GFP (13 μg/100 μl PBS), DCpep-GFP (1.3 μg/100 μl PBS) and commercial standard GFP (2.0 μg/100 μl PBS) respectively, at 37° C. for 1 hour. After PBS washing, B, T and mast cell pellets were stained with 1:3000 diluted DAPI and fixed with 2% paraformaldehyde. Then the cells were sealed on slides and examined by confocal microscopy. Live DCs were stained with 1:3000 diluted Hoechst and directly detected under the confocal microscope. For 293T, Pancreatic cells (PANC-1 and HPDE) and macrophage cells, eight-well chamber slides were used for cell culture at 37° C. for overnight. After incubated with purified CTB-GFP (8.8 μg/100 μl PBS), PTD-GFP (13 μg/100 μl PBS), DCpep-GFP (1.3 μg/100 μl PBS) and commercial standard GFP (2.0 μg/100 μl PBS) respectively, at 37° C. for 1 hour, washed in PBS and stained nuclei with 1:3000 DAPI. (FIG. 5B) Nuclear localization of PTD-GFP in human pancreatic ductal epithelial cells (HPDE). Green fluorescence shows GFP expression; blue fluorescence shows cell nuclei labeling with DAPI. The images were observed at 100× magnification. Scale bar represent 10 μm. All images studies have been analyzed in triplicate.

FIGS. 6A and 6B. Uptake of GFP fused with different tags by different human somatic and stem cell types. (FIG. 6A) In vitro evaluation of transformation of purified fused protein CTB-GFP, PTD-GFP, DCpep-GFP and GFP-Protegrin-1 (GFP-PG1) and GFP-Retrocyclin101 (GFP-RC101) in human pancreatic cells (PANC-1 and HPDE), human periodontal ligament stem cell (HPDLS), maxilla mesenchymal stem cells (MMS), human head and neck squamous cell carcinoma cells (SCC), retinal pigment epithelium cell (RPE), gingiva-derived mesenchymal stromal cells (GMSC), adult gingival keratinocytes (AGK) and osteoblast cell (OBC) with confocal microscopy. 2×10⁴ cells of human cell lines PANC-1, HPDE, HPDLS, MMS, SCC, RPE, GMSC, AGK and OBC were cultured in 8 well chamber slides (Nunc) at 37° C. for overnight, followed by incubation with purified GFP fusion proteins: CTB-GFP (8.8 μg), PTD-GFP (13 μs), DCpep-GFP (1.3 μg), GFP-PG1 (1.2 μg), GFP-RC101 (17.3 μg) in 100 μl PBS supplemented 1% FBS combined, respectively, at 37° C. for 1 hour. After washing wells with PBS for three times, cells were stained with 1:3000 DAPI at RT for 10 min and then fixed with 2% paraformaldehyde at RT for 10 min. For negative controls wells, cells were incubated with commercial GFP (2 μg) in PBS with 1% FBS or had no treatment. After 1 h of incubation, cells were washed three times with PBS. All fixed cells were imaged using confocal microscope. Green fluorescence shows GFP expression; blue fluorescence shows cell nuclei labeling with DAPI. The images were observed under 100× objective. Scale bar represent 10 μm. All images studies have been analyzed in triplicate. FIG. 6B: In vitro evaluation of transformation of purified fused protein CTB-GFP, PTD-GFP, DCpep-GFP, PG1-GFP and antimicrobial peptides (PG1-GFP and RC101-GFP) in human pancreatic cells (PANC-1 and HPDE), Human Periodontal Ligament stem cell (HPDLS), Maxilla Mesenchymal Stem cells (MMS), Human head and neck squamous cell carcinoma cells (SCC), Retinal pigment epithelium cell (RPE), Gingiva-derived mesenchymal stromal cells (GMSC), Adult gingival keratinocytes (AGK), Osteoblast cell (OBC) with confocal microscopy.

### DETAILED DESCRIPTION OF THE INVENTION

Targeted oral delivery of GFP fused with a GM1 receptor binding protein (CTB) or human cell penetrating peptide (PTD) or dendritic cell peptide (DCpep) was investigated. Presence of GFP⁺ intact plant cells between villi of ileum confirm their protection in the digestive system from acids/enzymes. Efficient delivery of GFP to gut-epithelial cells by PTD or CTB and to M cells by all these fusion tags confirm uptake of GFP in the small intestine. PTD fusion delivered GFP more efficiently to most tissues or organs than other two tags. GFP was efficiently delivered to the liver by all fusion tags, likely through the gut-liver axis. In confocal imaging studies of human cell lines using purified GFP fused with different tags, GFP signal of DCpep-GFP was only detected within dendritic cells. PTD-GFP was only detected within kidney or pancreatic cells but not in immune modulatory cells (macrophages, dendritic, T, B, or mast cells). In contrast, CTB-GFP was detected in all tested cell types, confirming ubiquitous presence of GM1 receptors. Such low-cost oral delivery of protein drugs to sera, immune system or non-immune cells should dramatically lower their cost by elimination of prohibitively expensive fermentation, protein purification cold storage/transportation and increase patient compliance.

### Definitions

As used herein, the terms “administering” or “administration” of an agent, drug, or peptide to a subject includes any route of introducing or delivering to a subject a compound to perform its intended function. The administering or administration can be carried out by any suitable route, including orally, intranasally, parenterally (intravenously, intramuscularly, intraperitoneally, or subcutaneously), rectally, or topically. Administering or administration includes self-administration and the administration by another.

As used herein, the terms “disease,” “disorder,” or “complication” refers to any deviation from a normal state in a subject.

As used herein, by the term “effective amount” “amount effective,” or the like, it is meant an amount effective at dosages and for periods of time necessary to achieve the desired result.

As used herein, the term “inhibiting” or “treating” means causing the clinical symptoms of the disease state not to worsen or develop, e.g., inhibiting the onset of disease, in a subject that may be exposed to or predisposed to the disease state, but does not yet experience or display symptoms of the disease state.

As used herein, the term “expression” in the context of a gene or polynucleotide involves the transcription of the gene or polynucleotide into RNA. The term can also, but not necessarily, involves the subsequent translation of the RNA into polypeptide chains and their assembly into proteins.

A “transgenic plant” refers to a plant whose genome has been altered by the introduction of at least one heterologous nucleic acid molecule.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

The terms “percent similarity”, “percent identity” and “percent homology” when referring to a particular sequence are used as set forth in the University of Wisconsin GCG software program.

The term “substantially pure” refers to a preparation comprising at least 50 60% by weight of a given material (e.g., nucleic acid, oligonucleotide, protein, etc.). More preferably, the preparation comprises at least 75% by weight, and most preferably 90 95% by weight of the given compound. Purity is measured by methods appropriate for the given compound (e.g. chromatographic methods, agarose or polyacrylamide gel electrophoresis, HPLC analysis, and the like).

A “replicon” is any genetic element, for example, a plasmid, cosmid, bacmid, phage or virus, that is capable of replication largely under its own control. A replicon may be either RNA or DNA and may be single or double stranded.

A “vector” is any vehicle to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The term “oligonucleotide,” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The phrase “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “promoter region” refers to the 5′ regulatory regions of a gene (e.g., 5′UTR sequences (e.g., psbA sequences, promoters (e.g., universal Prnn promoters or psbA promoters endogenous to the plants to be transformed and optional enhancer elements.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by calorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like.

The term “selectable marker gene” refers to a gene that when expressed confers a selectable phenotype, such as antibiotic resistance, on a transformed cell or plant. The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The term “DNA construct” refers to a genetic sequence used to transform plants and generate progeny transgenic plants. These constructs may be administered to plants in a viral or plasmid vector. However, most preferred for use in the invention are plastid transformation vectors. Other methods of delivery such as Agrobacterium T-DNA mediated transformation and transformation using the biolistic process are also contemplated to be within the scope of the present invention. The transforming DNA may be prepared according to standard protocols such as those set forth in “Current Protocols in Molecular Biology”, eds. Frederick M. Ausubel et al., John Wiley & Sons, 1995.

The phrase “double-stranded RNA mediated gene silencing” refers to a process whereby target gene expression is suppressed in a plant cell via the introduction of nucleic acid constructs encoding molecules which form double-stranded RNA structures with target gene encoding mRNA which are then degraded.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the basic and novel characteristics of the sequence. The term “tag,” “tag sequence” or “protein tag” refers to a chemical moiety, either an oligonucleotide, or more preferably a peptide or other chemical, that when added to another sequence, provides additional utility or confers useful properties, such as specifically targeting a protein of interest to the desired cell type. Such tags can also be useful for isolating and purifying the fusion proteins containing them. Protein tags such as those described herein or others commonly used in the art may be added to either the amino- or carboxy-terminus of the protein of interest.

An “immune response” signifies any reaction produced by an antigen, such as a viral or plant antigen, in a host having a functioning immune system. Immune responses may be either humoral in nature, that is, involve production of immunoglobulins or antibodies, or cellular in nature, involving various types of B and T lymphocytes, dendritic cells, macrophages, antigen presenting cells and the like, or both. Immune responses may also involve the production or elaboration of various effector molecules such as cytokines, lymphokines and the like. Immune responses may be measured both in in vitro and in various cellular or animal systems.

An “antibody” or “antibody molecule” is any immunoglobulin, including antibodies and fragments thereof, that binds to a specific antigen. Several of the drugs listed in Table 4 are antibodies. The term includes polyclonal, monoclonal, chimeric, and bispecific antibodies. As used herein, antibody or antibody molecule contemplates both an intact immunoglobulin molecule and an immunologically active portion of an immunoglobulin molecule such as those portions known in the art as Fab, Fab′, F(ab′)2 and F(v).

As used herein, a fusion peptide increases the ability of a protein to enter a cell by fusing with the cell membrane without requiring a specific receptor. Other fusion proteins can target cell receptors. Certain fusion proteins specifically target immune cells. Others target dendritic cells, such as DC peptide. Some fusion proteins are quite non specific and can be used to deliver therapeutic proteins to a variety of cell types of interest.

A plant remnant may include one or more molecules (such as, but not limited to, proteins and fragments thereof, minerals, nucleotides and fragments thereof, plant structural components, etc.) derived from the plant in which the protein of interest was expressed. Accordingly, a composition pertaining to whole plant material (e.g., whole or portions of plant leafs, stems, fruit, etc.) or crude plant extract would certainly contain a high concentration of plant remnants, as well as a composition comprising purified protein of interest that has one or more detectable plant remnants. In a specific embodiment, the plant remnant is rubisco.

In another embodiment, the invention pertains to an administrable composition for treating or preventing disease via administration of a therapeutic fusion protein produced in a plant chloroplast comprising a tag directing the therapeutic fusion protein to a target cell or tissue of interest. The composition comprises a therapeutically-effective amount of the fusion protein expressed by a plant and a plant remnant.

Methods, vectors, and compositions for transforming plants and plant cells are taught for example in WO 01/72959; WO 03/057834; and WO 04/005467. WO 01/64023 discusses use of marker free gene constructs.

Proteins expressed in accord with certain embodiments taught herein may be used in vivo by administration to a subject, human or animal in a variety of ways. The pharmaceutical compositions may be administered orally or parenterally, i.e., subcutaneously, intramuscularly or intravenously, though oral administration is preferred. Lists of therapeutic proteins of interest are provided in Example II.

Oral compositions produced by embodiments of the present invention can be administered by the consumption of the foodstuff that has been manufactured with the transgenic plant producing the plastid derived therapeutic fusion protein. The edible part of the plant, or portion thereof, is used as a dietary component. The therapeutic compositions can be formulated in a classical manner using solid or liquid vehicles, diluents and additives appropriate to the desired mode of administration. Orally, the composition can be administered in the form of tablets, capsules, granules, powders and the like with at least one vehicle, e.g., starch, calcium carbonate, sucrose, lactose, gelatin, etc. The preparation may also be emulsified. The active immunogenic or therapeutic ingredient is often mixed with excipients which are pharmaceutically acceptable and compatible with the active ingredient. Suitable excipients are, e.g., water, saline, dextrose, glycerol, ethanol or the like and combination thereof. In addition, if desired, the compositions may contain minor amounts of auxiliary substances such as wetting or emulsifying agents, pH buffering agents, or adjuvants. In a preferred embodiment the edible plant, juice, grain, leaves, tubers, stems, seeds, roots or other plant parts of the pharmaceuticalproducing transgenic plant is ingested by a human or an animal thus providing a very inexpensive means of treatment of or immunization against disease.

In a specific embodiment, plant material (e.g. lettuce, tomato, carrot, low nicotine tobacco material etc) comprising chloroplasts capable of expressing the therapeutic fusion protein, is homogenized and encapsulated. In one specific embodiment, an extract of the lettuce material is encapsulated. In an alternative embodiment, the lettuce material is powderized before encapsulation.

In alternative embodiments, the compositions may be provided with the juice of the transgenic plants for the convenience of administration. For said purpose, the plants to be transformed are preferably selected from the edible plants consisting of tomato, carrot and apple, among others, which are consumed usually in the form of juice.

According to another embodiment, the subject invention pertains to a transformed chloroplast genome that has been transformed with a vector comprising a heterologous gene that expresses a therapeutic fusion protein or peptide as disclosed herein.

Reference to the protein sequences herein relate to the known full length amino acid sequences as well as at least 12, 15, 25, 50, 75, 100, 125, 150, 175, 200, 225, 250 or 265 contiguous amino acids selected from such amino acid sequences, or biologically active variants thereof. Typically, the polypeptide sequences relate to the known human versions of the sequences.

Variants which are biologically active, refer to those, in the case of oral tolerance, that activate T-cells and/or induce a Th2 cell response, characterized by the upregulation of immunosuppressive cytokines (such as IL10 and IL4) and serum antibodies (such as IgG1), or, in the case of desiring the native function of the protein, is a variant which maintains the native function of the protein. Preferably, naturally or non-naturally occurring polypeptide variants have amino acid sequences which are at least about 55, 60, 65, or 70, preferably about 75, 80, 85, 90, 96, 96, or 98% identical to the full-length amino acid sequence or a fragment thereof. Percent identity between a putative polypeptide variant and a full length amino acid sequence is determined using the Blast2 alignment program (Blosum62, Expect 10, standard genetic codes). Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of polypeptide can be found using computer programs well known in the art, such as DNASTAR software. Whether an amino acid change results in a biologically active therapeutic fusion polypeptide can readily be determined by assaying for native activity, as described for example, in the specific Examples, below.

Reference to genetic sequences herein refers to single- or double-stranded nucleic acid sequences and comprises a coding sequence or the complement of a coding sequence for polypeptide of interest. Degenerate nucleic acid sequences encoding polypeptides, as well as homologous nucleotide sequences which are at least about 50, 55, 60, 65, 60, preferably about 75, 90, 96, or 98% identical to the cDNA may be used in accordance with the teachings herein polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of nucleic acid sequences which encode biologically active polypeptides also are useful polynucleotides.

Variants and homologs of the nucleic acid sequences described above also are useful nucleic acid sequences. Typically, homologous polynucleotide sequences can be identified by hybridization of candidate polynucleotides to known polynucleotides under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each homologous sequences can be identified which contain at most about 25-30% basepair mismatches. More preferably, homologous nucleic acid strands contain 15-25% basepair mismatches, even more preferably 5-15% basepair mismatches.

Species homologs of polynucleotides referred to herein also can be identified by making suitable probes or primers and screening cDNA expression libraries. It is well known that the Tm of a double-stranded DNA decreases by 1-1.5° C. with every 1% decrease in homology (Bonner et al., J. Mol. Biol. 81, 123 (1973). Nucleotide sequences which hybridize to polynucleotides of interest, or their complements following stringent hybridization and/or wash conditions also are also useful polynucleotides. Stringent wash conditions are well known and understood in the art and are disclosed, for example, in Sambrook et al., MOLECULAR CLONING: A LABORATORY MANUAL, 2^{nd} ed., 1989, at pages 9.50-9.51.

Typically, for stringent hybridization conditions a combination of temperature and salt concentration should be chosen that is approximately 12-20° C. below the calculated Tₘ of the hybrid under study. The Tₘ of a hybrid between a polynucleotide of interest or the complement thereof and a polynucleotide sequence which is at least about 50, preferably about 75, 90, 96, or 98% identical to one of those nucleotide sequences can be calculated, for example, using the equation of Bolton and McCarthy, Proc. Natl. Acad. Sci. U.S.A. 48, 1390 (1962):

Tm=81.5° C.−16.6(log 10[Na+])+0.41(% G+C)−0.63(% formamide)−600/l),

where l=the length of the hybrid in basepairs. Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C. The following materials and methods are provided to facilitate the practice of the present invention.

### Creation of Transplastomic Lines Expressing Different Tagged GFP Fusion Proteins

The transplastomic plants expressing CTB-GFP and PTD-GFP were created as described in previous studies [6, 34]. DC specific peptide, identified from screening of the Ph.D. 12-mer phage display library [26] was conjugated to the C-terminus of GFP and cloned into chloroplast transformation vector and transplastomic lines expressing DCpep-GFP were created and homoplasmic lines were confirmed using Southern blot assay as described previously [35]. Also, expression of GFP tagged proteins were confirmed by visualizing green fluorescence from the leaves of each construct under UV illumination.

### Lyophilization

The harvested mature leaves were stored at −80° C. and freeze-dried using lyophilizer (Genesis 35XL, VirTis SP Scientific) by which frozen and crumbled small pieces of leaves are subject to sublimation under the condition of vacuum (400 mTorr) and gradual augmentation of chamber temperature from −40° C. to 25° C. for 3 days. The lyophilized leaves were then ground in a coffee grinder (Hamilton Beach) at maximum speed 3 times (10 sec each). The powdered plant cells were stored under air-tight and moisture-free condition at room temperature with silica gel.

### Quantification of GFP Fusion Proteins and GM1 Binding Assay

The densitometry assay for quantification of GFP fusion proteins and GM1 ELISA assay were carried out according to previous method [14] except for using GFP standard protein (Vector laboratories MB-0752-100) and mouse monoclonal anti-GFP antibody (EMD MILLIPORE MAB3580). Non-denaturing Tris-tricine gel for identification of pentameric structure of CTB-GFP was performed according to previous study [36].

### Purification of Tag-Fused GFP Proteins

The protein purification of GFP fused peptides, PTD-GFP and DCpep-GFP, was performed by organic extraction/FPLC based method as described before [37]. Approximately 200 mg lyophilized plant cells were was homogenized in 10 ml of plant extraction buffer (100 mM NaCl, 10 mM EDTA, 200 mM Tris-Cl pH 8.0, 0.2% TritonX, 400 mM Sucrose, 2% v/v PMSF, 1 protease inhibitor tablet in 10 ml total volume). The homogenate was spun down after sonication and the supernatant was collected. The supernatant was transferred to a 50 ml falcon tube and subjected to organic extraction as performed previously [37]. The plant extract was treated with saturated ammonium sulfate to a final concentration of 70% in the extract. Then ¼th of the total extract volume of 100% ethanol was added, mixed vigorously for 2 min and then spun down. The resulting organic phase (upper phase) was collected to a fresh 50 ml falcon tube. To the remaining aqueous phase, 1/16th of total volume of 100% ethanol was added, shaken vigorously for 2 min and then spun down again. The organic phases from both the spins were pooled together and ⅓rd of total volume of 5 M NaCl and ¼th of the resulting volume of n-butanol was added and shaken vigorously for 2 min and spun down. The resulting organic extract layer (lower phase) at the bottom of the tube was collected and then desalted by running it through a 7 KDa MWCO desalting column (Thermo scientific zeba spin column 89893). The organic extract was loaded onto the desalting column and spun down as per manufacturer's instructions. Then the desalted organic extract (approximately 5 ml volume) was then loaded onto a FPLC column (LKB-FPLC purification system, Pharmacia; 48 mL column volume). During the purification process, the sample was washed with 3.5 column volumes of Buffer A (10 mM Tris HCl, 10 mM EDTA and 29 μm ammonium sulfate set at pH 7.8) with 20% ammonium sulfate saturation. The column was then subjected to step wise increase in Buffer B (10 mM Tris HCl, 10 mM EDTA sulfate set at pH 7.8) to elute the GFP fusion. The protein was detected by measurement of absorbance at 280 nm, which corresponded to a single peak that was plotted on a recorder. The fraction corresponding with the peak was collected in a single tube having a total volume of 9 ml. The purified fraction was then dialyzed in 2 L of 0.01×PBS thrice and then lyophilized (Labconco lyophilizer). The lyophilized purified GFP fusions were then quantified by western blot/densitometric method.

For the purification of CTB-GFP, lyophilized leaf materials (400 mg) were resuspended in 20 ml of extraction buffer (50 mM Na—P, pH7.8; 300 mM NaCl; 0.1% Tween-20; 1 tb of EDTA-free protease inhibitor cocktail). The resuspension was sonicated and then centrifuged at 10,000 rpm for 10 min at 4° C. The supernatant was combined with 1 ml of His60 Ni resin (Clonetech, 635657), and purification was performed according to manufacturer's instructions.

### Purity Measurement and Coomassie Staining

Total protein of purified from each GFP tag fraction was quantified using Bradford assay then as described in quantification section, densitometry assay was carried out to quantify the amount of GFP fusion protein in the fractions. Then the purity was evaluated by calculating the percentage of the amount of GFP fusion proteins to the total amount of protein obtained from Bradford assay. A non-denaturing SDS-PAGE was also performed in order to check fluorescence of the GFP fused proteins by running through a 10% SDS gel under non-denaturing conditions.

### Evaluation of GFP Expression

GFP presence in sera and tissues were quantified by our in house GFP ELISA. As our previously described [6, 16], the blood and tissue samples were collected at 2 and 5 hours after the last oral gavage and serum were stored at −80° C. Tissues were homogenized in RIPA buffer and supernatants were collected for GFP ELISA assay. Our in house ELISA protocol was established and the standards were calibrated based on GFP ELISA kit (AKR121; Cell Biolab). Briefly, 96-well Maxisorp plates (Nunc) were coated overnight at 4° C. with goat polyclonal GFP antibody (2.5 mg/ml, Rockland) in coating buffer (PH 9.6). The plates were blocked in PBS with 3% BSA for 2 hrs at 37° C. Serial 2-fold dilutions of sera in PBS with 1% BSA were added in duplicate and incubated overnight at 4° C. The plate was then incubated with biotin-conjugated rabbit polyclonal anti-GFP 1:5000 (Rockland) overnight at 4° C., followed by addition of 1:2000 diluted streptavidin peroxidases (Rockland). After further incubation for 1 hr at 37° C., plates were washed and substrate solution was added and incubated for 10 min at RT. The reaction was stopped by adding 100 μl of 2N sulfuric acid per well, and absorbance was measured using an ELISA reader at 450 nm. The results are shown as average±SEM.

### Mice and Oral Delivery Experiments

Eight-week-old female C57BL/6 mice (Jackson Laboratories, Bar Harbor, Mich.) were randomly divided into four groups (n=6 per group) and orally gavaged with each lyophilized bioencapsulated CTB-GFP, PTD-GFP and DCpep-GFP plant cells, 20 mg/per mouse/day for 3 consecutive days. All lyophilized materials were suspended in 200 μl PBS. On the 3rd day, blood samples were collected 2 and 5 hrs after the last oral gavage. At the 5 hr time point, all mice were sacrificed, and organs (liver, kidney, lung, brain, tibialis anterior muscle) were harvested and stored at −80° C. A control group (n=6) was fed with untransformed lyophilized plant cells. Mice were housed in the animal facility of University of Florida and University of Pennsylvania under controlled humidity and temperature conditions, and the experiments were performed in accordance with the guidelines of Institutional Animal Care and Use Committees.

### Immunofluorescent Staining

As our previously described [8, 16], C57BL/6 mice were orally fed with GFP expressing plant cells twice by 2 hr interval. Two hours after last feeding, mice were sacrificed, live and intestines were removed. The intestine cut open longitudinally and washed by PBS, then rolled up and fixed overnight in 4% paraformaldehyde at 4° C. Liver tissue was also fixed similarly. Subsequently, fixed tissues were further incubated in 30% sucrose in PBS at 4° C. and embedded in OCT. Serial sections were cut to a thickness of 10 μm.

For analysis of GFP expression, sections were permeabilized with 0.1% Triton X-100, and blocked with 5% donkey serum in PBS for 30 min, followed by incubation with rabbit anti-GFP antibody at 1:1000 (ab290, Abeam) overnight at 4° C. The sections were then incubated for 30 min with Alexa Fluor 488 Donkey Anti-Rabbit IgG (Jackson ImmunoResearch) or with rhodamine-labeled Ulex europaeus agglutinin (UEA-1; Vector Labs; 10 μg/mL) for 10 min before being washed and mounted with or without DAPI (4, 6 diamidino-2-phenylindole). Images were captured using a Nikon Eclipse 80i fluorescence microscope and Retiga 2000R digital camera (QImaging) and analyzed with Nikon Elements software.

### Uptake of Purified Tag-Fused GFP Proteins by Human Cell Lines

To determine uptake of three tags, CTB, PTD and DC peptide in immune modulatory cells, mature dendritic cell, human T cell (Jurkat cell), human B cell (BCBL1), macrophage cells (mØ), mast cells and non-immune modulatory cells, human kidney cells (293T), human pancreatic epithelioid carcinoma cells (PANC-1) and human pancreatic ductal epithelial cells (HPDE), were cultured and used for in vitro transformation of purified tag-fused proteins. Cells (2×10⁴) were incubated in 100 μl PBS supplemented 1% FBS combined with purified CTB (8.8 μg), PTD (13.5 μg) and DCpep (1.3 μg) fused protein, respectively, incubated at 37° C. for 1 hour. After PBS washing, cell pellets were stained with 1:3000 diluted DAPI and fixed with 2% paraformaldehyde at RT for 10 min. Cells were then sealed on the slides with cytoseal and examined by confocal microscopy. For live image study, after incubation with purified GFP fused proteins, dendritic cells were loaded on glass bottom microwell dishes (MatTek) and observed under confocal microscopy followed by nuclei staining with DAPI. For 293T, PANC-1 and macrophage cells, cells were cultured in 8 well chamber slides (Nunc) at 37° C. for overnight, followed by incubation with purified CTB-GFP, PTD-GFP and DCpep-GFP (same as above) at 37° C. for 1 hour. After washing wells with PBS, cells were stained with 1:3000 DAPI and fixed with 2% paraformaldehyde at RT for 10 min. For negative controls groups, cells incubated with commercial GFP (2 μg) in PBS with 1% FBS or had no treatment. After 1 h of incubation, cells were washed once with PBS. Live non-fixed cells were imaged using confocal microscope.

To determine the uptake efficiency of purified GFP fusion proteins in different human cell lines, the number of cells showing GFP signals was counted and represented as percentage of over total number of cells observed. A total of 15-20 images were recorded for each cell line in three independent samples the under confocal microscope at 100× magnification.

The following examples are provided to illustrate certain embodiments of the invention. They are not intended to limit the invention in any way.

### Example I

### Uptake of GFP Fused with Different Tags by Human Cells

In this study, we investigate oral administration of plant cells expressing GFP fused with different tags in chloroplasts and evaluate cellular targeting and their bio-distribution. Delivery of PTD, DCpep, and CTB fusions across the gut epithelium utilizing distinct pathways result in systemic delivery, bio-distribution, and, perhaps most importantly, distinct patterns of uptake by non-immune or immune modulatory cells. These peptides could be used to deliver therapeutic proteins to sera, immune modulatory cells or specific tissues.

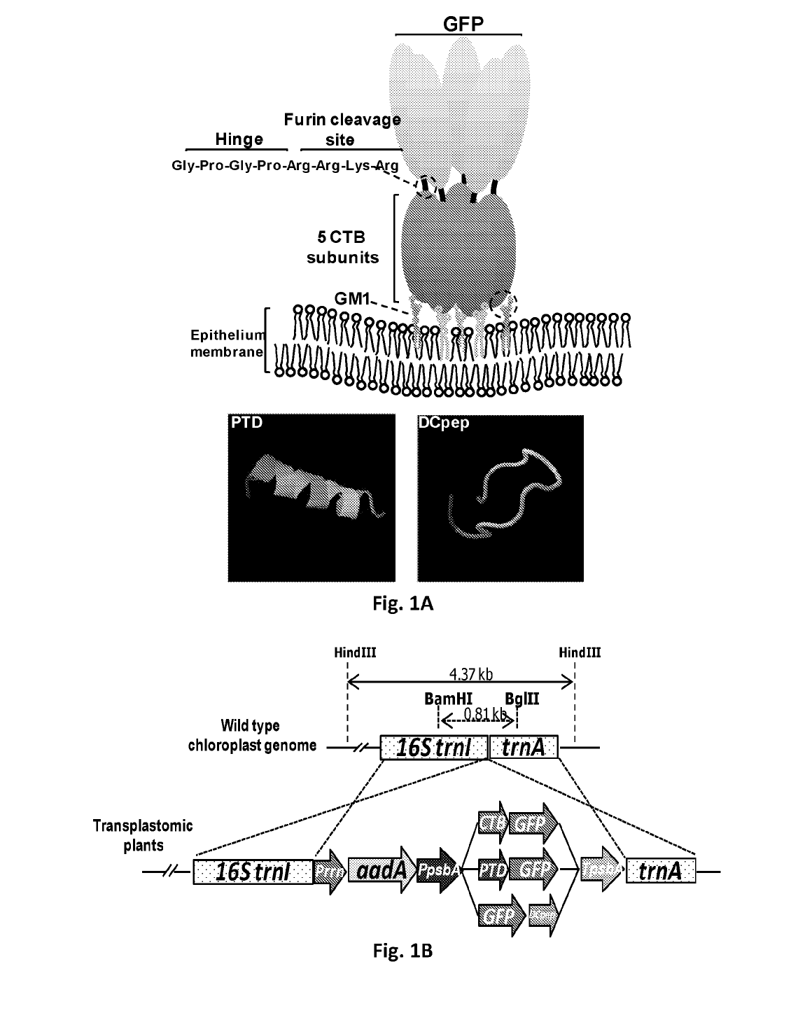

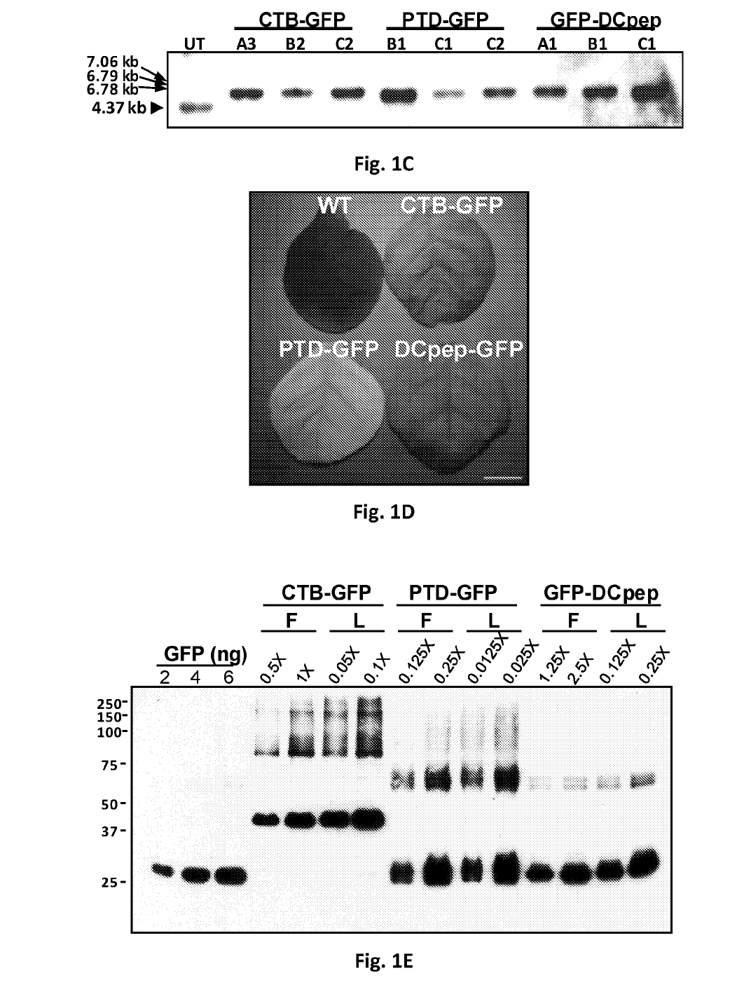

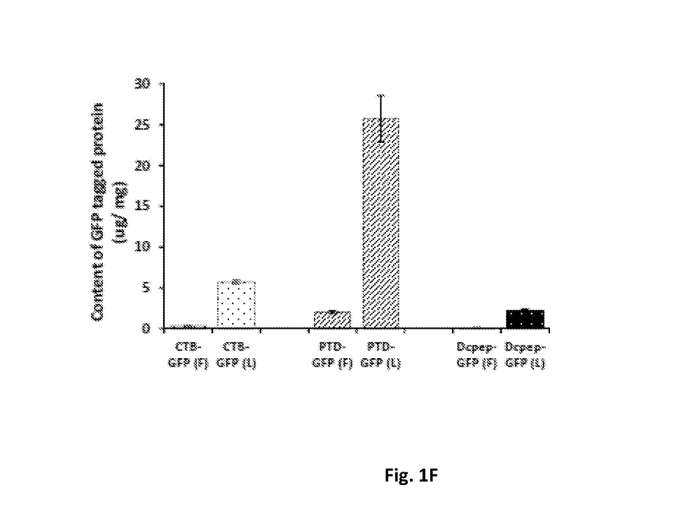

Three distinct transmucosal carriers were tested. Interaction of CTB pentamer to GM1 receptor has been well studied as shown in the FIG. 1A. The penta-saccharide structure of GM1 receptor interacts with amino acids of CTB via hydrogen bonds [38]. In contrast, mechanisms of structures shown for PTD (FIG. 1A) or DCpep (FIG. 1A) has not been fully investigated. Secondary structures of cell-penetrating peptides (CPP) have been studied by circular dichroism (CD). Such peptides interact with negatively charged phospholipid vesicles leading to induction of secondary structures. Small uni-lamellar vesicles (SUVs) are used in such studies. However, the exact role of any secondary structure of CPPs in relation to translocation process is difficult to define. It has been shown that CPPs change their structure from alpha helix to beta sheets depending upon experimental conditions, even in simple model systems. So, they are described as “chameleons” in changing their structure, rapidly adapting to membrane environment (39). Therefore, secondary structures were not investigated in our studies but we used interative threading assembly refinement program (I-TASSER) to predict computational 3-D structures [40]. In the FIG. 1A, the representative models were chosen based on the calculation of parameters such as confidence score (C-score), high-resolution models with root mean square deviation (RMSD) value, and template modeling score (TM-score). For example, if the TM-score, assessing topological similarity of first I-TASSER model to corresponding structure in Protein Data Bank (PDB) library, is greater than 0.5, the predicted topology is correct [40]. From the predicted model, TM-score of PTD was 0.60±0.14 and that of DCpep was 0.58±0.14. In predicted structures, while PTD has helical structure, DCpep shows a loop structure. As expected, there is no similarity between the two peptides.

All three tags were fused to the green fluorescent protein (smGFP) to evaluate their efficiency and specificity. CTB (MIKLKFGVFFTVLLS SAYAHGTPQNITDLCAEYHNTQIHTLNDKIF SYTESLAGKREMAI ITFKNGATFQVEVPGSQHIDSQKKAIERMKDTLRIAYLEAKVEKLCVWNNKTPHAIAAIS MAN; SEQ ID NO: 1) was fused with GFP at the N-terminus via furin cleavage site, Pro-Arg-Ala-Arg-Arg (SEQ ID NO: 2) [6]. Sixteen amino acid (RHIKIWFQNRRMKWKK; SEQ ID NO: 3) derived from pancreatic and duodenal homeobox factor-1 (PDX-1) [41] is fused at the N-terminus with GFP and is referred to as PTD-GFP in this study. For nuclear targeting, additional localization signals are required. Six amino acids (RH, RR, and KK) of the 16 aa-PTD are critical for nuclear localization of PDX-1(42). Human dendritic cell specific peptide ligand (FYPSYHSTPQRP (SEQ ID NO: 4) identified from screening of 12-mer phage display library [26] is fused to the C-terminus of GFP. Both PTD and DC-Peptide were engineered without the furin cleavage site to study entry as well as tissue distribution. All these three fusion constructs were cloned into the chloroplast transformation vectors (pLD) which were used to transform chloroplasts as described in the material methods section.

To create plants expressing GFP fusion proteins, tobacco chloroplasts were transformed using the biolistic particle delivery system. As seen in the FIG. 1B, each tag-fused GFP is driven by identical regulatory sequences—the psbA promoter and 5′ UTR regulated by light and the transcribed mRNA is stabilized by 3′ psbA UTR. The psbA gene is the most highly expressed chloroplast gene and therefore psbA regulatory sequences are used for transgene expression in our lab [7, 35]. To facilitate the integration of the expression cassette into chloroplast genome, two flanking sequences, isoleucyl-tRNA synthetase (trnI) and alanyl-tRNA synthetase (trnA) genes, flank the expression cassette, which are identical to the native chloroplast genome sequence. The emerging shoots from selection medium were investigated for specific integration of the transgene cassette at the trnI and trnA spacer region and then transformation of all chloroplast genomes in each plant cell (absence of untransformed wild type chloroplast genomes) by Southern blot analysis with the Dig-labeled probe containing the trnI and trnA flanking sequences (FIG. 1C). As seen in FIG. 1C, HindIII-digested gDNAs from three lines of each GFP plant showed transformed large DNA fragments at 7.06, 6.79 and 6.78 kbp, for CTB-GFP, PTD-GFP and DCpep-GFP, respectively, when hybridized with the probe and absence of the untransformed smaller fragment (4.37 kbp). Thus, stable integration of three different GFP expression cassettes and homoplasmy of chloroplast genome with transgenes were confirmed. In addition, by visualizing the green fluorescence under UV light, GFP expression of was phenotypically monitored (FIG. 1D).

To scale up the biomass of each GFP tagged plant leaf material, each homoplasmic line was grown in a temperature- and humidity-controlled automated greenhouse. Fully grown mature leaves were harvested in late evenings to maximize the accumulation of GFP fusion proteins driven by light-regulated control sequences. To further increase the content of the fusion proteins on a dry weight basis, frozen leaves were freeze-dried at −40° C. under vacuum. In addition to the concentration effect of proteins, lyophilization increased shelf life of therapeutic proteins expressed in plants more than one year at room temperature [13]. Therefore, in this example, lyophilized and powdered plant cells expressing GFP-fused tag proteins were used for oral delivery to mice. Immunoblot assay for the GFP fused tag proteins showed identical size proteins in fresh and 4-month old lyophilized leaves (FIG. 1E), confirming stability of fusion proteins during lyophilization and prolonged storage at room temperature. In the immunoblot image, in addition to monomers of 39.5 kDa, 29.2 kDa, and 28.3 kDa for CTB-GFP, PTD-GFP, and DCpep-GFP, respectively, dimers were also detected for PTD-GFP (58.4 kDa) and DCpep-GFP (56.6 kDa) (FIG. 1E). Homodimerization is one of GFP physio-chemical features, which occurs in solution and in crystals. The contacts between the monomers are very tight due to extensive interactions which are composed of a core of hydrophobic side chains from each monomer and a number of hydrophilic contacts [43]. Also, CTB monomer can be self-assembled to pentameric structure which is very stable and resistant heat and denaturants due to the intersubunit interactions within pentameric structure, which is mediated by hydrogen bonds, salt bridges and hydrophobic interactions [44].

GFP protein concentration in powdered lyophilized leaf materials was 5.6 ug/mg, 24.1 ug/mg and 2.16 ug/mg for CTB-GFP, PTD-GFP, and DCpep-GFP, respectively (FIG. 1C). GFP protein concentration after lyophilization increased 17.1-, 12.7-, and 18.8-fold for CTB-GFP, PTD-GFP, and DCpep-GFP, respectively (FIG. 1F). Removal of water from fresh leaves by lyophilization is attributed to the reduction of weight by 90-95%. This effect is then manifested as 10-20 fold increase of protein per gram of dry leaves [13, 15, 45].

GFP Uptake in Different Tissues after Oral Delivery of Plant Cells

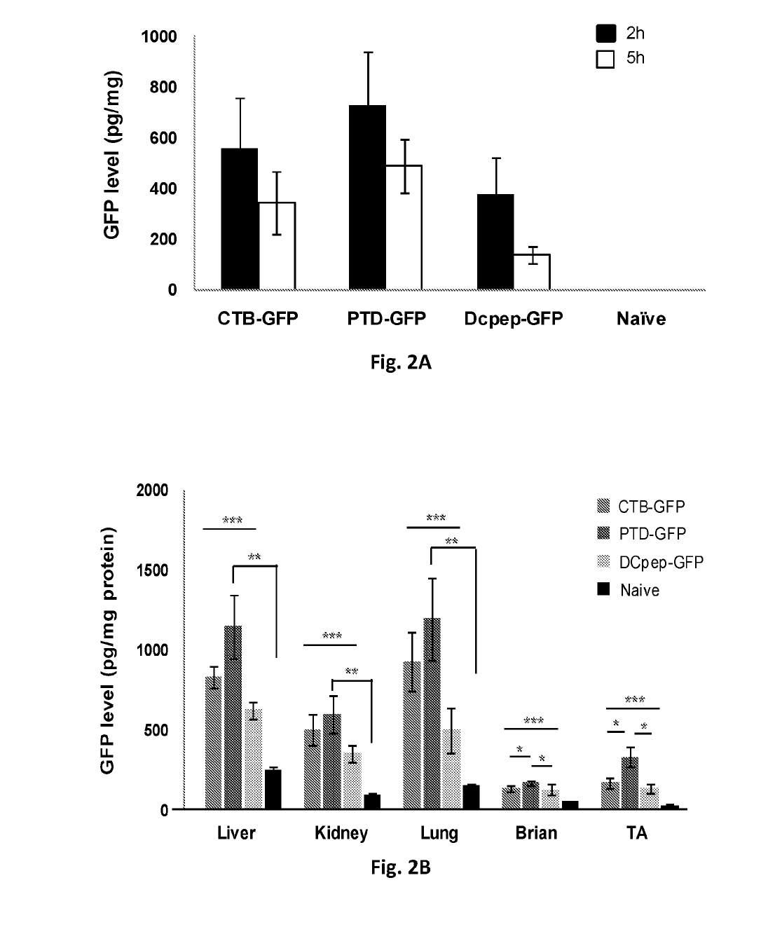

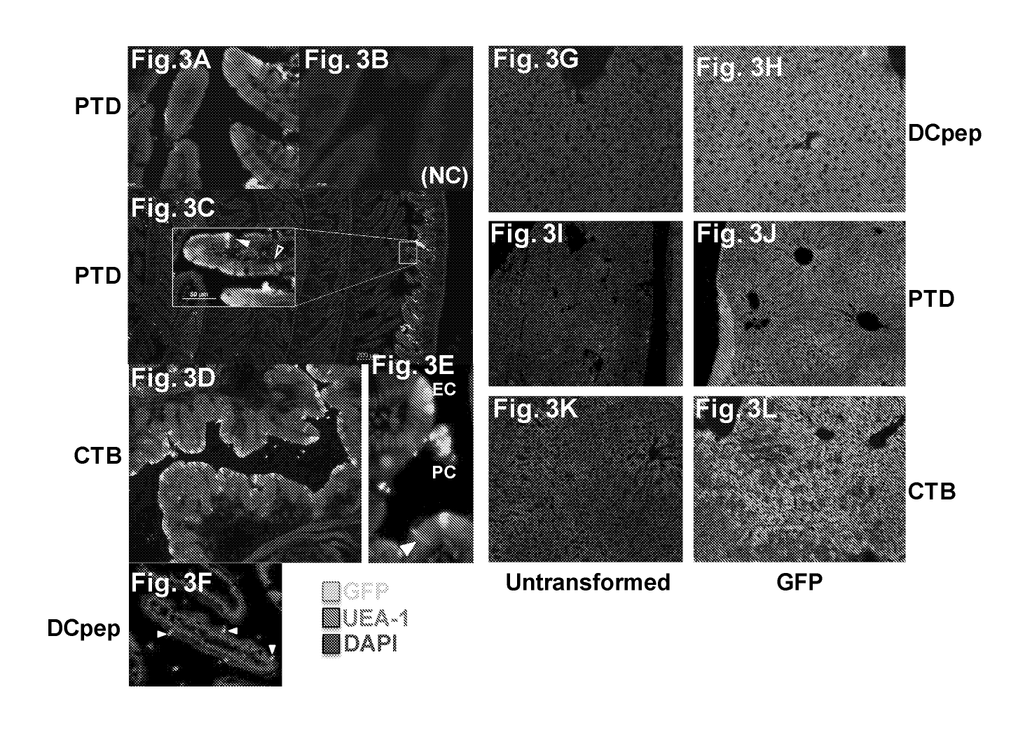

For oral delivery, lyophilized plant cells (20 mg) were rehydrated in uniform volume (200 μl) and similar durations. Dispersed plant cells do not vary in their size because mature plant cells are uniform in size. As seen in FIG. 2, the bio-distribution of GFP does not show any significant variations. After oral delivery of lyophilized plant cells expressing GFP (fused with PTD or CTB or DC peptide), systemic GFP levels were higher in PTD-GFP fed animals than any other tags tested (FIG. 2A). Biodistribution to liver and lung was substantially higher than other tissues (skeletal muscle, kidney). GFP levels in these tissues were consistently highest for PTD fusion protein (FIG. 2B). Immunohistochemical studies using GFP-specific antibody offered further insight into the route of delivery. As shown in FIG. 3A (3C insert), sensitive method of detection of GFP using Alexa Flour 488 labeled secondary antibody revealed that the PTD tag directed some GFP uptake by gut epithelial cells. No GFP was detected when no primary antibody was used or when tissues from mice fed with untransformed tobacco cells (FIG. 3B). In order to more readily find areas of gut where plant cells are located and antigen uptake may be observed, the small intestine was rolled up prior to fixation, so that proximal and distal portions were visible on the same slide (FIG. 3C). Presence of plant cells expressing GFP in between villi of ileum (FIGS. 3C and E) offers first direct proof for protection of plant cells from the digestive system. More widespread delivery of GFP to epithelial cells was also seen when using the CTB tag (FIG. 3D) and this is due to efficient targeting of the GM1 receptor with by CTB pentamers. In addition to delivery to epithelial cells, we also found evidence for uptake of GFP by M cells (solid arrows in FIG. 3C-F) by all fusion tags. Again, these observations provide direct evidence for uptake of proteins in the upper gut after their lysis in the gut. FIG. 3E in particular illustrates the presence of GFP⁺ plant cells (“PC”) of CTB-GFP transplastomic plants near the site of delivery of released GFP to epithelial cells (“EC”) and M cells (solid arrow) of the ileum. For DCpep-GFP, no GFP⁺ epithelial cells were observed. However, we found examples of co-localization of GFP and M cells (FIG. 3F), suggesting that systemic delivery of DCpep-GFP is possible due to transport from the gut lumen via M cells. GFP delivered with all three tags showed accumulation in the liver (FIGS. 3H, J and L), as expected because the blood can carry antigens from the gut to the liver via the portal vein (“gut-liver-axis”).

Purification of GFP Fused with Different Tags

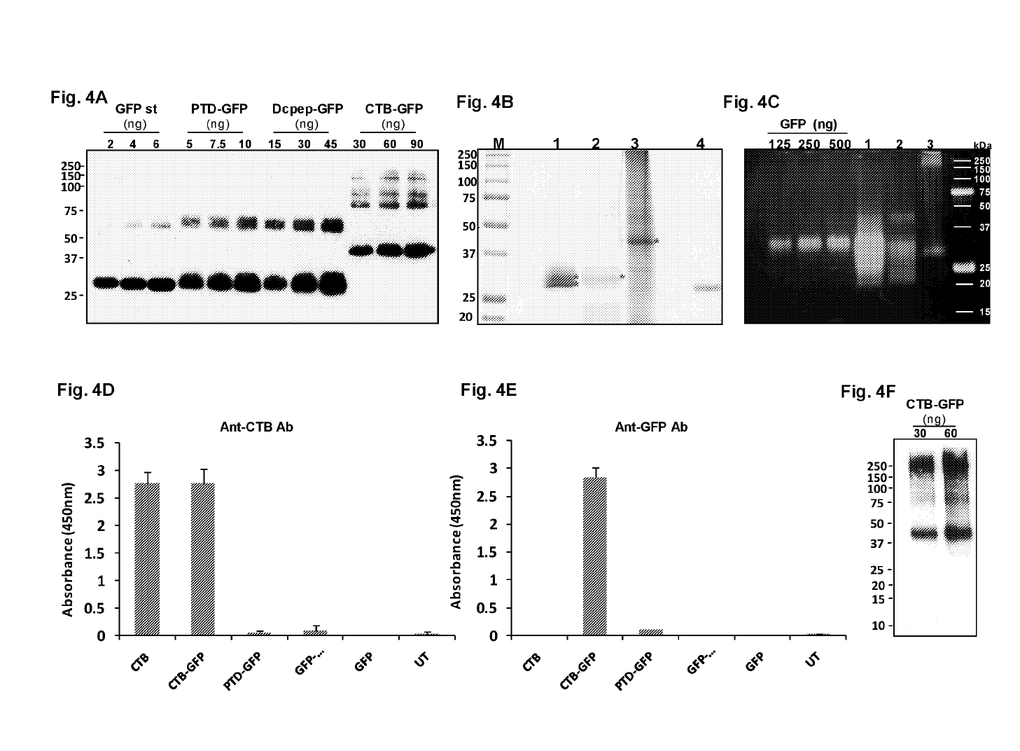

To study uptake of GFP by different cell types, the GFP fusion proteins were purified using toyopearlbutyl column for PTD-GFP and DCpep-GFP, and Ni²⁺ column for CTB-GFP. To examine the purity, densitometry was done using western blots and GFP standard (FIG. 4A). The purity of each tag fused GFP was ˜95% for PTD-GFP, ˜52% for DCpep-GFP, and ˜13% for CTB-GFP. The variation in purity levels is attributed to the differences in the expression levels of each tag which is reflected on the recovered GFP fusion proteins after purification. Because proteins are purified based on hydrophobic interaction, other hydrophobic proteins could be present in the purified fractions. The purification of CTB-GFP was done with affinity Ni²⁺ column. Histidine cluster is generated when pentameric structure of CTB is formed, then the imidazole rings in the histidine cluster interact with Ni²⁺[46]. The low purity of CTB-GFP is due to less stringent wash step, but increasing the stringency was accompanied with higher loss of the fused protein.

Purified GFP fusion proteins in SDS-PAGE and Coomassie stained gels showed distinct bands for each fusion protein at expected sizes, 29.2 kDa, 28.3 kDa and 39.5 kDa, for PTD-GFP, DCpep-GFP and CTB-GFP, respectively, (FIG. 4B). In case of PTD-GFP, there are two bands around at the expected size, which were 29.2 and 28.3 kDa. It has been reported that the C-terminal tail (His-Gly-Met-Asp-Glu-Tyr-Lys) of GFP is quite susceptible to proteolytic cleavage by carboxypeptidases and by nonspecific proteases including proteinase K and pronase, and various isoforms, caused by partial proteolytic cleavage, generated by ion exchange chromatography, isoelectric focusing, and native gel electrophoresis [47]. In order to determine GFP fluorescence, non-denaturing SDS-PAGE was performed where the fluorescence intensity was strongest in PTD-GFP (Lane 1) (FIG. 4C). In DCpep-GFP (Lane 2) 3 distinct proteins were seen with the top band most likely representing dimerized GFP [47] and the same was observed in PTD-GFP although it was of much higher intensity. The CTB-GFP fusion showed a set of larger fluorescence bands (Lane 3) starting from 190 kDa which corresponds to the pentamerization of CTB fused GFP and the other higher bands likely representing multimers. At the same lane, a smaller fragment slightly lower than the GFP standards was observed which could be a differently folded product similar to what was observed in PTD-GFP and DCpep-GFP.

To evaluate proper formation of pentameric structure of purified CTB-GFP, GM1 binding assay was performed with anti-CTB and anti-GFP antibody. It is well known that the pentameric structure of CTB has strong binding affinity to ganglioside GM1 receptors which are found ubiquitously on the surface of mammalian cells [48]. As seen in FIG. 4D, only CTB and CTB-GFP showed binding affinity, indicating complex formation between CTB and GM1. Also, the interaction of GM1-CTB-GFP was reconfirmed using anti-GFP antibody. Only CTB-GFP can be detected (FIG. 4E). To further confirm the pentameric structure of CTB-GFP, the purified CTB-GFP was run on the modified Tris-tricine gels under the non-denaturing conditions [36] and probed using anti-CTB antibody. The expected pentameric CTB-GFP form was detected at ˜200 kDa along with the monomeric form at 39.5 kDa (FIG. 4F). It is likely that the monomer is dissociated from the oligomeric structure during the run due to SDS, which was added in the gel and electrophoresis buffer. Therefore, the CTB-GFP fusion protein formed the pentameric structure and retained ability to bind to GM1 receptors, but there is no GM1 binding affinity for PTD-GFP or DCpep-GFP fusion protein.

Uptake of GFP Fused with Different Tags by Human Immune and Non-Immune Cells

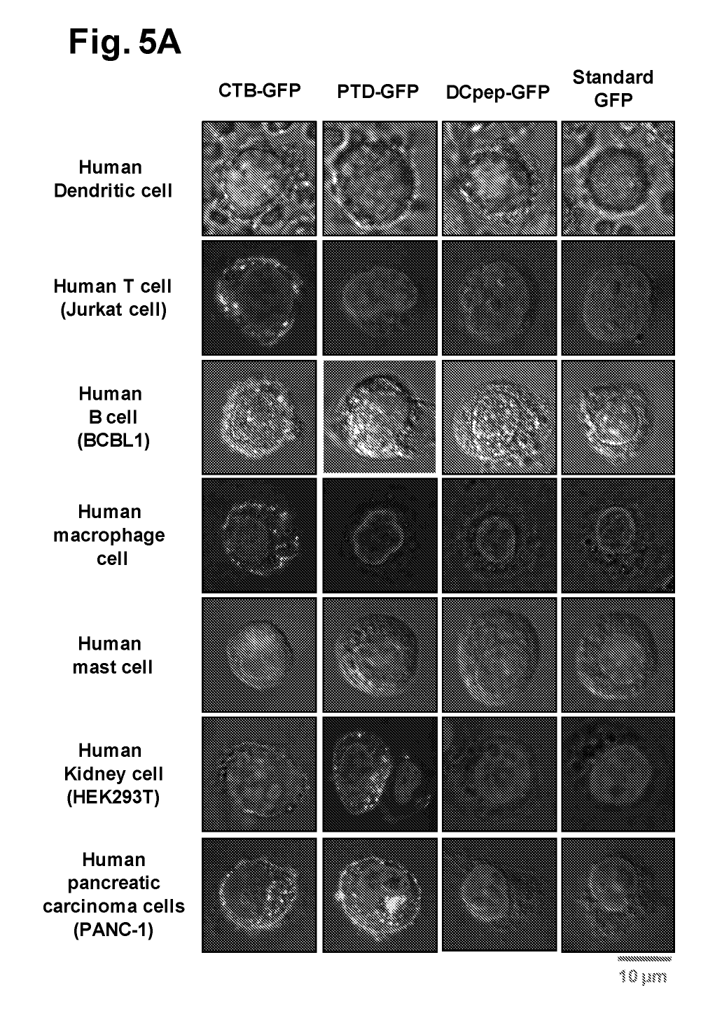

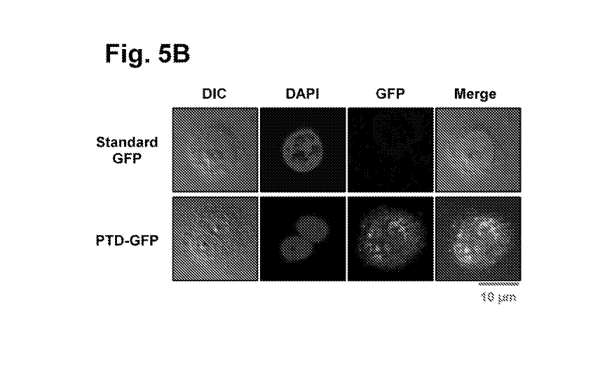

Purified GFP fusion proteins were incubated with human cultured cells. Blood monocyte-derived mature DC, T cells (Jurkat cell), B cells (BCBL1), differentiated macrophages and mast cells were cultured for in vitro studies. Human kidney cells (HEK293T) and human pancreatic epithelioid carcinoma cells (PANC-1) were tested in parallel as examples of non-immune cells. Cells (2×10⁴) were incubated with purified CTB, PTD and DC target peptide fused GFP for one hour at 37° C. Upon incubation with DCpep-GFP, intracellular GFP signal was detected only in DCs and not for any of the other cell type, confirming its specificity (FIG. 5A). PTD-GFP entered kidney cells or pancreatic cells but failed to enter any of the immune modulatory cells (FIG. 5A). PTD sequence was derived from PDX1 that induces insulin expression in pancreatic cells, and the exogenous PDX1 could penetrate mouse insulinoma cell line and activated insulin gene [41]. As expected, strong GFP signal was observed from PANC-1 cells when incubated with purified PTD-GFP. Also, PTD-GFP was observed in nucleus of the pancreatic ductal epithelial cells (FIG. 5B). The cell penetrating ability of PTD was also evident in human kidney cell line (FIG. 5A). In sharp contrast, GFP signals were detected in all cell types upon incubation with CTB-GFP, consistent with ubiquitous presence of GM1 receptors (FIG. 5A). Bone marrow-derived murine mast cells (a cell type that plays important roles in wound healing, defense to pathogens, and allergic reactions) showed no internalization of GFP delivered by DCpep or PTD, while CTB fused GFP was efficiently taken up (FIG. 5A). Although only one representative image is presented here, the uptake studies were performed in triplicates and 15-20 images for each cell line were recorded under confocal microscopy. CTB-GFP was observed in 70-92% of all the cell types examined and the variations are due to differences in cell density resulting in lower availability of GFP for their uptake. In case of PTD-GFP, no uptake (0%) was observed in any other cell type except kidney and pancreatic cells. DCpep-GFP was not observed in any other cell type (0%) except in dendritic cells (Table 1). This study offers specificity to deliver protein drugs to sera, immune system or specific organs or tissues, thereby facilitating further advancement of this novel concept.

TABLE 1
Uptake efficiency of purified GFP fusion proteins in human cell lines.
The relative delivery efficiency of GFP to human cell lines
by three different tags was compared by counting the number
of cells showing GFP signals under confocal microscope at 100x
magnification. A total of 15-20 images were observed for
each cell line. All cell lines were examined in triplicate.

| | CTB-GFP | PTD-GFP | | DCpep-GFP |
|---|---|---|---|---|
| Human Dendritic | 70.4% (19/27) | 0% | (0/12) | 83% (10/12) |
| cell | | | | |
| Human T cell | 91.7% (11/12) | 0% | (0/18) | 0% (0/13) |
| Human B cell | 87.5% (14/16) | 0% | (0/14) | 0% (0/10) |
| Human macrophage | 91.7% (11/12) | 0% | (0/12) | 0% (0/12) |
| cell | | | | |
| Human mast cell | 91.7% (11/12) | 0% | (0/12) | 0% (0/11) |
| Human Kidney cell | 83.3% (12/13) | 83.3% | (10/12) | 0% (0/10) |
| Human pancreatic | 89.5% (17/19) | 81.2% | (9/11) | 0% (0/12) |
| cells | | | | |

### 4. Discussion

In our previous publications [6-16], we have shown that plant cell-derived proteins fused with CTB can be delivered across the intestinal epithelium for various applications, including delivery of functional proteins to the circulatory system and of protein antigens to the immune system (for oral tolerance induction or as a booster vaccine). This is possible because of binding of CTB pentamers to the GM1 receptors on the surface of gut epithelial and M cells, followed by transcytosis. However, GM1 is widely expressed by many different cell types and it is difficult to target to specific cell types. Therefore, in this study we sought to develop alternative tags that result in either more cell type-specific and/or more efficient transmucosal delivery. We chose DCpep as an example for specific delivery to professional antigen presenting cells and were able to confirm its specificity. PTD not only delivers the GFP cargo more effectively to the circulation via penetration of intestinal epithelial cells (FIGS. 3 A and 3C) but totally avoids delivery to immune cells (FIG. 5A). In our opinion, data presented here on PTD are paradigm shift in drug delivery, in sharp contrast to previous assumptions that they are non-specific.

Given recent disagreements in the literature about mechanisms of cell penetration by PTDs (macropinocytosis vs direct transfer) and about effects of PTDs vs the cargo they carry (49), this is a very timely contribution to this field. Our data show that the body's immune system can be excluded from protein drug delivery by specific choice of PTD, while efficiently delivering to other cell types and circulation. Thus, this study provides a new approach for increasing the specific targeting of therapeutics to selected tissue types using preselected PTDs. Combined with sequence and structural knowledge, custom-designed PTDs can be generated. We used defined in vitro systems to demonstrate the differences in protein transduction between different tags as a function of the target cell type. This approach can also be employed to assess the immunological consequences, when disease-specific antigens instead of GFP and corresponding animal models are employed.

To reveal routes by which the GFP fusion proteins cross the intestinal epithelial layer and delivery to circulation and biodistribution to other organs, the immunostaining studies of small intestine tissue sections after oral delivery of leaf materials expressing three GFP tag proteins were carried out. CTB was confirmed to be targeted to GM1 receptors on epithelial cells and by M cells. Our study found that PTD is able to penetrate non-immune cells, which explains why this tag also transfers GFP to epithelial cells. In contrast, DC peptide is a specific ligand to dendritic cells and therefore should not target epithelial cells. However, our data show that uptake by M cells is a mechanism of entry for DCpep fusions, thus explaining how systemic delivery of DCpep-GFP is possible via the oral route. Dendritic cells are also directly targeted by DCpep through their protrusions between epithelial cells. However, the amounts of gut luminal antigen sampled by this mechanism are too small to be visualized.

We expect that protein antigens released from CTB after transcytosis are taken up by DC (and to a lesser extent by macrophages) in the gut immune system (as we have extensively published) and that DCpep-GFP translocated by M cells is also taken up by DC. However, for initial studies of interactions with immune cells, we utilized a more sensitive and defined in vitro approach using cultured human cells.

CTB fusion delivered GFP to all tested tissues and cell types including non-immune and immune cells. It is well established that CTB specifically binds with GM1 ganglioside and a variety of CTB-fused proteins expressed in chloroplasts in our lab also showed the strong binding affinity to GM1 [6, 8, 9, 13-16]. CTB travels retrograde through the trans-Golgi Network into the endoplasmic reticulum (ER) for cell entry once CTB binds with GM1, enriched in membrane lipid rafts of intestinal epithelial cells [50, 51]. In fact, CTB has been widely used as a probe to quantitatively study GM1 and its cellular and subcellular distribution [52]. The use of CTB as a transmucosal carrier can facilitate the transportation of conjugated proteins into circulation through its strong binding affinity to GM1 and the large mucosal area of human intestine (approximately 1.8-2.7 m² against body weight [53]. Up to 15,000 CTB molecules can bind to one intestinal epithelial cell at a time [54] and the GM1 receptor turns over rapidly on the cell surface [55]. Furthermore, GM1 gangliosides are also found in the plasma membranes of many other cell types, with particular abundance in the nervous system and retina [56, 57], thus directing efficient uptake of CTB fusion protein in these cells.

Our strategy of oral tolerance induction to autoantigens and to therapeutic proteins used in replacement therapy of genetic disorders (such as hemophilia and lysosomal storage disorders) has in part relied on efficient targeting of gut epithelial cells with CTB followed by transmucosal delivery and proteolytic cleavage, resulting in the release of the antigen from the CTB tag and uptake by DCs [7, 9, 10, 11, 33,]. However, DCs and macrophages also directly sample antigen in the gut lumen, and M cells may shuttle intact antigen across the epithelium to areas rich in DCs. Our new data show that CTB fusions are efficiently taken up by DC, macrophages, and other immune cells, providing an additional explanation for the effectiveness of CTB fused antigens in plant-based immune modulatory protocols.

### Specific Targeting of Dendritic Cells by DCpep Ideal for Delivery to the Immune System

Here, we introduce an alternative and more specific peptide sequence with high potential for immunotherapy. DCpep specifically targets DCs but not any other immune cells or non-immune cells. To assess translational implications, we mostly used human immune cells to differentiate targeting characteristics of the fusion tags. DCpep only delivered intact GFP antigen to DCs but not any other APCs or immune cells or non-immune cells. Consistent with this finding, DCpep-GFP failed to target gut epithelial cells in vivo. Systemic delivery most likely resulted from uptake by M cells. Going forward, one can now design immune tolerance and vaccine protocols based on specific delivery to DC, which have critical functions in Treg induction and immune stimulation, depending on activation signals.

As we know, the delivery of antigens to lymph node is quite important for immunotherapy. However, this aspect cannot be directly demonstrated by GFP signal because proteins taken up by DC in the lamina propria or Peyer's patches are fragmented into small peptides and loaded onto MHC molecules by the time the DC have migrated to the lymph nodes for presentation of antigen to T cells. In agreement with the notion that mesenteric lymph nodes (MLN) are critical in their response to ingested antigens, we recently demonstrated increases in different DC subsets and induction of Treg in the MLNs of mice that received oral delivery of transplastomic plant cells [10].

### PTD is Ideal for Efficient Systemic Delivery Via the Oral Route Excluding the Immune System

While CTB fusions effectively target the gut immune system and are thus useful for tolerance induction, an alternative strategy to avoid immune complications is to minimize interactions with the immune system. The protein transduction domain of PDX-1 exhibited unique selectivity in the transfer of GFP to different cell types. PTD-GFP entirely failed to deliver antigen to APCs and lymphocytes but was able to transfer GFP to non-immune cells (including gut epithelial cells in vivo). Since myeloid and lymphoid cells are hematopoietic cells, it is possible that PDX-1 fails to transduce this specific cell lineage. PDX-1 induces insulin expression upon protein transduction via macropinocytosis, a specialized form of endocytosis that is distinct from receptor-mediated uptake [59, 60]. Macropinocytosis is also major mechanism of uptake for macromolecules in kidney, so the observation of GFP signals in HEK293T after incubation with purified PTD-GFP could be the consequence of the endocytosis induce by PTD. Lack of GFP signal in immune cells after incubation with PTD-GFP cannot be explained by enhanced degradation after uptake but rather reflects a failure of protein transduction of these cells because i) there was also a lack of binding to the cell surface, and ii) the PTD of HIV tat, which also utilizes the macropinocytosis mechanism, readily delivers intact GFP into human DC and other APCs by the PTD of HIV tat [61-63]. PTD derived from PDX-1 clearly displays a distinct selectivity for cellular transduction, possibly related to surface properties of the target cell membrane. Although both PTDs enter the cell by macropinocytosis, their amino acid sequences are very different, which is likely to affect cell surface binding. While infection of lymphocytes is a critical step of the HIV life cycle, insulin expression needs to be tightly regulated and responsive to environmental stimuli [64], which may in part account for the selectivity of PTD from PDX-1. At the same time, PTD-GFP was superior in vivo for systemic protein delivery, which can be exploited for therapies that require certain protein levels in the blood, such as in our published examples of treatment of hypertension and hormone or cytokine therapies [9, 11, 14, 15]. Interestingly, despite marked differences in systemic delivery, CTB-, DCpep-, and PTD-tags all resulted in very similar GFP antigen levels in the liver, as evidenced by immunohistochemistry and more quantitatively by ELISA. Links between responses in the gut and the liver have long been known and are often referred to as the “gut-liver axis”. Upon uptake by the gut, antigen can traffic to the liver indirectly via migratory DC that is routed through the mesenteric lymph node. Alternatively, the blood can carry antigens from the gut to the liver via the portal vein. Given the broad distribution of quantifiable levels of GFP in the liver, the latter explanation seems more likely for our delivery system. The data suggest that the liver takes up the orally delivered antigen to a level of saturation that is less dependent on the tag.

### Unique Advantages of Delivery of Proteins Bioencapsulated in Plant Cells

Lyophilization of plant cells has several advantages. The freeze-dried powdered leaves can be stored at room temperature for years eliminating expensive cold storage and transportation which are required for injectable protein drugs [13, 65]. Also, concentration effect of the therapeutic protein is increased facilitating 10-20 fold reduction in the size of capsules containing lyophilized plant cells. Freeze drying technology is widely used to preserve protein drugs by the pharmaceutical industry, including preservation of blood clotting factors. So, freeze drying process doesn't denature proteins. Indeed, we have repeatedly shown that freeze drying preserves proper folding and disulfide bonds (11, 13-16, 66). Upon oral delivery, lyophilized plant cells reach the intestine, and the bioencapsulated proteins are released by gut microbes through digestion of plant cell wall. That time, the released proteins as well as plant cell walls can be degraded by gut microbes. However, it is possible that the gut microbiome is enriched by anaerobic bacteria that release more enzymes to degrade plant cell wall than protein degradation. Bacteria inhabiting the human gut have indeed evolved to utilize complex carbohydrates in plant cell wall and are capable of utilizing almost all plant glycans [4, 5]. Our previously published work identified enzymes that are required to breakdown plant cell wall [67, 68]. Delivery of several functional proteins show that they are either protected in the gut lumen or adequate quantities of protein drugs are released that survive gut lumen proteases.

DCpep-GFP content was found to be the lowest among three fusion proteins, 2.16 ug/mg, which is 10 times lower than that of PTD-GFP. Generally, chloroplast expression of foreign protein can reach very high level, up to 70% of total leaf proteins [7] due to high copy number of chloroplast genome. However, expression level varies based on protein, N-terminal fusions, proteolytic cleavage and stability. In this study, all the chimeric genes were driven by the psbA promoter and psbA 5′UTR, and stabilized psbA 3′UTR. Since the GFP sequence is also same among all three constructs, the contributing factors that affect the difference of the expression level could be due to the N-terminal sequence of the fusion constructs. In contrast to PTD and CTB tag, DCpep was fused to C-terminal of GFP. Therefore, one possible explanation for lower level of GFP expression of DCpep fusion is inadequate protection of the N-terminus.

### CONCLUSIONS

As illustrated above, bioavailability of oral delivery of protein drugs expressed in genetically modified plant cells is now emerging as a new concept for inducing tolerance against autoimmune disorders [7] or to eliminate toxicity of injected protein drugs [8-10] or deliver functional blood proteins to treat diabetes [12, 13], hypertension [14], protection against retinopathy [15] or removal of plaques in Alzheimer's brain [16]. These novel approaches should improve patient compliance in addition to significantly lowering the cost of healthcare as seen in the diabetes study in which oral delivery was as effective as injectable delivery to lower blood glucose levels using insulin or exendin-4 [12, 13].

This study has enabled utilization of different fusion tags to deliver either to immune modulatory cells or non-immune cells or directly to sera without interfering with the immune system. This opens up the potential for low cost oral delivery of proteins to enhance or suppress immunity or functional proteins to regulate metabolic pathways.

The cost of protein drugs now exceeds GDP of >75% of countries, making them unaffordable. This is because of their production in prohibitively expensive fermenters, purification, cold storage/transportation, short shelf life and sterile delivery methods. Using green fluorescent protein (GFP) as a model, we demonstrate in this study that plant cells protect GFP from the digestive system and release it into the gut lumen where they are absorbed by epithelial cells. Based on the delivery tag fused to GFP, they reach the circulatory system, immune cells or non-immune cells, specific organs or tissues. Such low cost oral delivery of protein drugs should increase patient compliance and dramatically lower their cost by elimination of currently used prohibitively expensive processes/injections.

### REFERENCES

[1] Walsh, G. Biopharmaceutical benchmarks 2014. Nature Biotechnology 2014; 32: 992-1000.
[2] Jin S, Daniell H. Engineered chloroplast genome just got smarter. Trends in Plant Science 2015; 20: 622-640.
[3] Chan H T, Daniell H. Plant-made vaccines against human infectious diseases—Are we there yet? Plant Biotechnol J 2015; 13: 1056-1070.
[4] Martens E C, Lowe E C, Chiang H, Pudlo N A, Wu M, McNulty N P, et al. Recognition and degradation of plant cell wall polysaccharides by two human gut symbionts. PLoS Biol 2011; 9, e1001221
[5] Flint H J, Bayer E A, Rincon M T, Lamed R. White B A. Polysaccharide utilization by gut bacteria: potential for new insights from genomic analysis. Nat Rev Microbiol 2008; 6: 121-131.
[6] Limaye A, Koya V, Samsam M, Daniell H. Receptor-mediated oral delivery of a bioencapsulated green fluorescent protein expressed in transgenic chloroplasts into the mouse circulatory system. FASEB J 2006; 20:959-961.
[7] Ruhlman T, Ahangari R, Devine A, Samsam M, Daniell H. Expression of cholera toxin B-proinsulin fusion protein in lettuce and tobacco chloroplasts—oral administration protects against development of insulitis in non-obese diabetic mice. Plant Biotechnol J 2007; 5: 495-510.
[8] Verma D, Moghimi B, LoDuca P A, Singh H D, Hoffman B E, Herzog R W, et al. (2010) Oral delivery of bioencapsulated coagulation factor IX prevents inhibitor formation and fatal anaphylaxis in hemophilia B mice. Proc Natl Acad Sci USA 2010; 107: 7101-7106.
[9] Sherman A, Su J, Lin S, Wang X, Herzog R W, Daniell H. Suppression of inhibitor formation against FVIII in a murine model of hemophilia A by oral delivery of antigens bioencapsulated in plant cells. Blood 2014; 124: 1659-1668.
[10] Wang X, Su J, Sherman A, Rogers G L, Liao G, Hoffman B E, et al. Plant-based oral tolerance for hemophilia results from a complex immune regulatory mechanism including LAP⁺ CD4⁺ T cells. Blood 2015; 125: 2418-2427.
[11] Su J, Sherman A, Doerfler P A, Byrne B J, Herzog R W, Daniell H. Oral delivery of Acid Alpha Glucosidase epitopes expressed in plant chloroplasts suppresses antibody formation in treatment of Pompe mice. Plant Biotechnol J. 2015; 13: 1023-1032.
[12] Boyhan D, Daniell H (2011) Low-cost production of proinsulin in tobacco and lettuce chloroplasts for injectable or oral delivery of functional insulin and C-peptide. Plant Biotechnol. J 2011; 9: 585-598.
[13] Kwon K C, Nityanandam R, New J S, Daniell H. Oral delivery of bioencapsulated exendin-4 expressed in chloroplasts lowers blood glucose level in mice and stimulates insulin secretion in beta-TC6 cells. Plant Biotechnol J 2013; 11:77-86
[14] Shenoy V, Kwon K C, Rathinasabapathy A, Lin 51, Jin G, Song C, et al. Oral delivery of Angiotensin-converting enzyme 2 and Angiotensin-(1-7) bioencapsulated in plant cells attenuates pulmonary hypertension. Hypertension 2014; 64: 1248-1259.
[15] Shil P K, Kwon K C, Zhu P, Verma A, Daniell H, Li Q. Oral Delivery of ACE2/Ang-(1-7) Bioencapsulated in Plant Cells Protects against Experimental Uveitis and Autoimmune Uveoretinitis. Molecular Therapy 2014; 22: 2069-2082.
[16] Kohli N, Westerveld D R, Ayache A C, Verma A, Shil P, Prasad T, et al. Oral Delivery of Bioencapsulated Proteins Across Blood-Brain and Blood-Retinal Barriers. Molecular Therapy 2014; 22: 535-546.
[17] Zahid M, Robbins P D. Protein transduction domains: applications for molecular medicine. Curr Gene Ther 2012; 12: 374-380.
[18] Khaja K, Robbins P. Comparison of Functional Protein Transduction Domains Using the NEMO Binding Domain Peptide. Pharmaceuticals 2010; 3: 110-124.
[19] Embury J, Klein D, Pileggi A, Ribeiro M, Jayaraman S, Molano R D, et al. Proteins linked to a protein transduction domain efficiently transduce pancreatic islets. Diabetes 2001; 50: 1706-1713.
[20] Schwarze S R, Ho A, Vocero-Akbani A, Dowdy S F. In vivo protein transduction: delivery of a biologically active protein into the mouse. Science 1999; 285: 1569-1572
[21] Mills C D. M1 and M2 Macrophages: Oracles of Health and Disease. Critical Reviews in immunology. 2012; 32: 463-488.
[22] Banchereau J, Steinman R M. Dendritic cells and the control of immunity. Nature 1998; 392: 245-252.
[23] Ingulli E, Mondino A, Khoruts A, Jenkins M K. In vivo detection of dendritic cell antigen presentation to CD4+ T cells. J Exp Med 1997; 185: 2133-2141.
[24] Luther S A, Gulbranson-Judge A, Acha-Orbea H, Maclennan I C M. Viral superantigen drives extrafollicular and follicular B differentiation leading to virus-specific antibody production. J Exp Med 1997; 185: 551-562.
[25] Kudo S, Matsuno K, Ezaki T, Ogawa M. A novel migration pathway for rat dendritic cells from the blood: Hepatic sinusoids-lymph translocation. J Exp Med 1997; 185: 777-784.
[26] Curiel T J, Morris C, Brumlik M, Landry S J, Finstad K, Nelson A, et al. Peptides identified through phage display direct immunogenic antigen to dendritic cells. J Immunol 2004; 172: 7425-7431.
[27] Banchereau J, Palucka A K. Dendritic cells as therapeutic vaccines against cancer. Nat Rev Immunol 2005; 5: 296-306.
[28] Celluzzi C M, Mayordomo J I, Storkus W J, Lotze M T, Falo L D Jr. Peptide-pulsed dendritic cells induce antigen-specific CTL-mediated protective tumor immunity. J Exp Med 1996; 183: 283-287.
[29] Kraehenbuhl J P, Corbett M. Immunology. Keeping the gut microflora at bay. Science 2004; 303: 1624-1625.
[30] von Boehmer H. Oral tolerance: is it all retinoic acid? J Exp Med 2007; 204: 1737-1739.
[31] Weiner H L. Oral tolerance: immune mechanisms and treatment of autoimmune diseases. Immunol Today 1997; 18: 335-343.
[32] Annacker O, Coombes J L, Malmstrom V, Uhlig H H, Bourne T, Johansson-Lindbom B, et al. Essential role for CD103 in the T cell-mediated regulation of experimental colitis. J Exp Med 2005; 202: 1051-1061.
[33] Biswas M, Sarkar D, Kumar S R, Nayak S, Rogers G L, Markusic D M, et al. Synergy between rapamycin and FLT3 ligand enhances plasmacytoid dendritic cell-dependent induction of CD4+CD25+FoxP3+Treg. Blood 2015; 125: 2937-2947.
[34] Kwon K C, Verma D, Jin S, Singh N D, Daniell H. Release of proteins from intact chloroplasts induced by reactive oxygen species during biotic and abiotic stress. PLoS One. 2013c;8:e67106.
[35] Verma D, Samson N P, Koya V, Daniell H. A protocol for expression of foreign genes in chloroplasts. Nat Protoc 2008; 3: 739-758.
[36] Haider S R, Sharp B L, Reid H J. A comparison of Tris-glycine and Tris-tricine buffers for the electrophoretic separation of major serum proteins. J Sep Sci 2011; 34: 2463-2467.
[37] Lee S, Li B, Jin S, Daniell H. Expression and characterization of antimicrobial peptides Retrocyclin-101 and Protegrin-1 in chloroplasts to control viraland bacterial infections. Plant Biotechnol J 2011; 9: 100-115.
[38] Sanchez J, Holmgren J. Cholera toxin—a foe & a friend. Indian J Med Res 2011; 133:153-163.
[39] Magzoub M, Eriksson L E, Graslund A. Conformational states of the cell-penetrating peptide penetratin when interacting with phospholipid vesicles: effects of surface charge and peptide concentration. Biochim Biophys Acta. 2002; 1563: 53-63.
[40] Roy A, Kucukural A, Zhang Y. I-TASSER: a unified platform for automated protein structure and function prediction. Nature Protocols 2010; 5: 725-738.
[41] Noguchi H, Kaneto H, Weir G C, Bonner-Weir S. PDX-1 protein containing its own antennapedia-like protein transduction domain can transduce pancreatic duct and islet cells. Diabetes 2003; 52: 1732-1737.
[42] Hessabi B, Ziegler P, Schmidt I, Hessabi C, Walther R. The nuclear localization signal (NLS) of PDX-1 is part of the homeodomain and represents a novel type of NLS. Eur J Biochem. 1999; 263:170-177.
[43] Phillips G N Jr. Structure and dynamics of green fluorescent protein. Curr Opin Struct Biol 1997; 7: 821-827.
[44] Miyata T, Oshiro S, Harakuni T, Taira T, Matsuzaki G, Arakawa T, et al. Physicochemically stable cholera toxin B subunit pentamer created by peripheral molecular constraints imposed by de novo-introduced intersubunit disulfide crosslinks. Vaccine 2012; 30: 4225-4232.
[45] Lakshmi P S, Verma D, Yang X, Lloyd B, Daniell H. Low cost tuberculosis vaccine antigens in capsules: expression in chloroplasts, bio-encapsulation, stability and functional evaluation in vitro. PLoS One 2013; 8:e54708.
[46] Davoodi-Semiromi A, Schreiber M, Nalapalli S, Verma D, Singh N D, Banks R K, et al. Chloroplast-derived vaccine antigens confer dual immunity against cholera and malaria by oral or injectable delivery. Plant Biotechnol J 2010; 8: 223-242.
[47] William W W. Biochemical and physical properties of green fluorescent protein. In: Chalfie M, Kain S R (eds). Green fluorescent protein, 2nd edn. Hoboken, N.J. 2006; pp 39-65.
[48] Daniell H, Lee S B, Panchal T, Wiebe P O. Expression of the native cholera toxin B subunit gene and assembly as functional oligomers in transgenic tobacco chloroplasts. J Mol Biol 2001; 311: 1001-1009.
[49] Durzyńska J, Przysiecka L, Nawrot R, Barylski J, Nowicki G, Warowicka A, Musidlak O, Goździcka-Józefiak A. Viral and other cell-penetrating peptides as vectors of therapeutic agents in medicine. J Pharmacol Exp Ther. 2015; 354: 32-42.
[50] Wernick N L, Chinnapen D J, Cho J A, Lencer W I. Cholera toxin: an intracellular journey into the cytosol by way of the endoplasmic reticulum. Toxins (Basel) 2010; 2: 310-325.
[51] Cho J A, Chinnapen D J, Aamar E, to Welscher Y M, Lencer W I, Massol R. Insights on the trafficking and retro-translocation of glycosphingolipid-binding bacterial toxins. Front Cell Infect Microbiol 2012; 2: 51
[52] Critchley D R, Kellie S, Streuli C H, Patel B, Ansell S, Pierce E, et al. The use of cholera toxin as a probe to study the organisation of ganglioside GM1 in membranes. Prog Clin Biol Res 1982; 102 pt A: 397-407.
[53] Wilson J P. Surface area of the small intestine in man. Gut 1967; 8: 618-621.
[54] Holmgren J, Lönnroth I, Mãnsson J, Svennerholm L. Interaction of cholera toxin and membrane GM1 ganglioside of small intestine. Proc. Natl. Acad. Sci. USA 1975; 72: 2520-2524.
[55] Fishman P H, Bradley R M, Hom B E, Moss J. Uptake and metabolism of exogenous gangliosides by cultured cells: effect of choleragen on the turnover of GM1. J Lipid Res 1983; 24: 1002-1011.
[56] Hadjiconstantinou M, Neff N H. GM1 ganglioside: in vivo and in vitro trophic actions on central neurotransmitter systems. J Neurochem 1998; 70: 1335-1345.
[57] Yu R K, Tsai Y T, Ariga T. Functional roles of gangliosides in neurodevelopment: an overview of recent advances. Neurochem Res 2012; 37: 1230-1244.
[58] Yu J, Langridge W H. A plant-based multicomponent vaccine protects mice from enteric diseases. Nat Biotechnol 2001; 19: 548-552
[59] Noguchi H, Matsushita M, Matsumoto S, Lu Y F, Matsui H, Bonner-Weir S, et al. Mechanism of PDX-1 protein transduction. Biochem Biophys Res Commun 2005; 332: 68-74.
[60] Noguchi H, Matsumoto S, Okitsu T, Iwanaga Y, Yonekawa Y, Nagata H, et al. PDX-1 protein is internalized by lipid raft-dependent macropinocytosis. Cell Transplant 2005; 14: 637-645.
[61] Kaplan I M, Wadia J S, Dowdy S F. Cationic TAT peptide transduction domain enters cells by macropinocytosis. J Control Release 2005; 102: 247-253.
[62] Batchu R B, Moreno A M, Szmania S M, Bennett G, Spagnoli G C, Ponnazhagan S, et al. Protein transduction of dendritic cells for NY-ESO-1-based immunotherapy of myeloma. Cancer Res 2005; 65: 10041-10049.
[63] Su Y, Zhang A H, Li X, Owusu-Boaitey N, Skupsky J, Scott D W. B cells “transduced” with TAT-fusion proteins can induce tolerance and protect mice from diabetes and EAE. Clin Immunol 2011; 140: 260-267.
[64] Calder P C, Dimitriadis G, Newsholme P. Glucose metabolism in lymphoid and inflammatory cells and tissues. Curr Opin Clin Nutr Metab Care 2007; 10: 531-540.
[65] Kwon K C, Verma D, Singh N D, Herzog R, Daniell H. Oral delivery of human biopharmaceuticals, autoantigens and vaccine antigens bioencapsulated in plant cells. Adv Drug Deliv Rev 2013; 65: 782-799.
[66] Su J, Zhu L, Sherman A, Wang X, Lin S, Kamesh A, Norikane J H, Streatfield S J, Herzog R W, Daniell H. Low cost industrial production of coagulation factor IX bioencapsulated in lettuce cells for oral tolerance induction in hemophilia B. Biomaterials. 2015; 70: 84-93
[67] Agrawal P, Verma D, Daniell H. Expression of Trichoderma reesei 0-mannanase in tobacco chloroplasts and its utilization in lignocellulosic woody biomass hydrolysis. PLoS One. 2011; 6: e29302.
[68] Verma D, Jin S, Kanagaraj A, Singh N D, Daniel J, Kolattukudy P E, Miller M, Daniell H. Expression of fungal cutinase and swollenin in tobacco chloroplasts reveals novel enzyme functions and/or substrates. PLoS One. 2013; 8: e57187.

### Example II

### Evaluation of Uptake of GFP Fused with Different Tags: CTB-GFP, PTD-GFP, PG1-GFP and RC101-GFP in Additional Human Cell Lines

To expand the types of cells that can be successfully targeted using the protein therapeutics described herein, we assessed both human somatic cell lines and stem cell lines were used. With the goal of producing targeted delivery of therapeutic proteins to benign and malignant tissues, six cell lines were selected. These lines included normal and cancerous cell lines from human dental, head and neck, and soft tissue. To develop functional protein-based treatments for developmental defects and for immature/stem cell tumors, three human stem cell lines were selected.

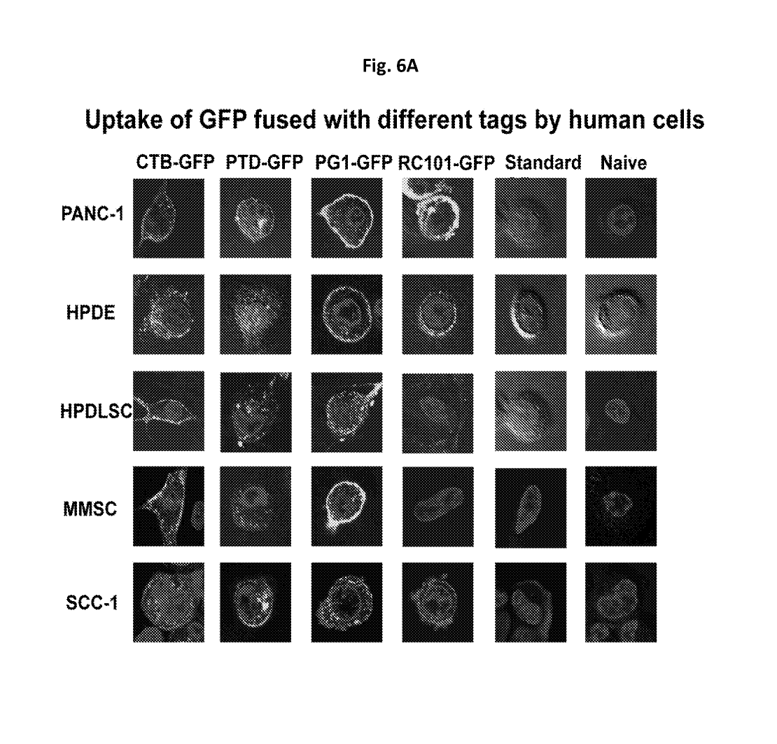

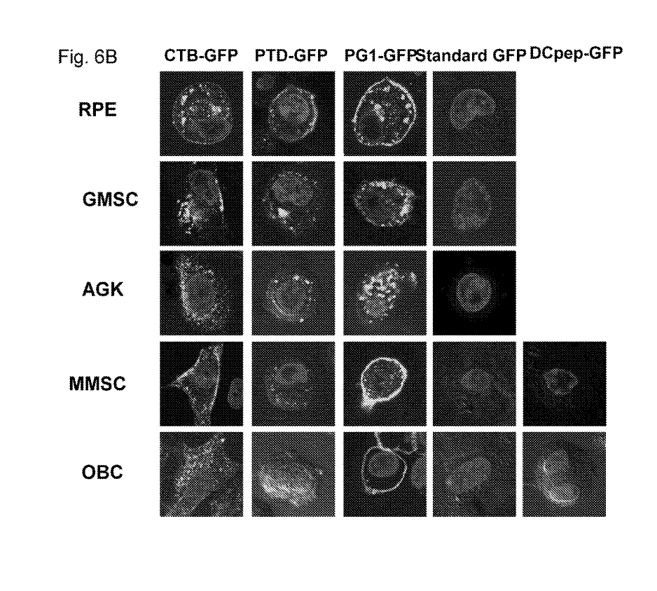

FIGS. 6A and 6B show the results obtained with different cell types and tags were studied. These human somatic cells included pancreatic cancer cells (PANC-1); Human pancreatic ductal epithelial cells (HPDE); Human head and neck squamous cell carcinoma cells (SCC-1); Retinal pigment epithelium cells (RPE); Adult gingival keratinocytes (AGK); Osteoblasts (OBC). Certain types of human stem cells were also assessed. These included Human periodontal ligament stem cells (HPDLSC); Maxillar mesenchymal stem cells (MMSC) and Gingiva-derived mesenchymal stromal cells (GMSC).

CTB, PTD and DC-peptide fusion tags are described in Example I. Protegrin (PG) and retrocyclin (RC) are antimicrobial peptides and are described in detail in Lee et al., Plant Biotechnology Journal 9: 100-115 (2011).

Protegrin 1:
(SEQ ID NO: 5)
Arg-Gly-Gly-Arg-Leu-Cys-Tyr-Cys-Arg-Arg-Arg-Phe-
Cys-Val-Cys-Val-Gly-Arg
Protegrin 2:
(SEQ ID NO: 6)
Arg-Gly-Gly-Arg-Leu-Cys-Tyr-Cys-Arg-Arg-Arg-Phe-
Cys-Val-Cys-Val

Protegrin-3 substitutes a glycine for an arginine at position 4 and it also has one less positive charge. Protegrin-4 substitutes a phenylalanine for a valine at position 14 and sequences are different in the β-turn. This difference makes protegrin-4 less polar than others and less positively charged. Protegrin-5 substitutes a proline for an arginine with one less positive charge.

Additional retrocyclin variants are provided in PCT/US2002/012353 which is incorporated by reference herein.

### Results

### PANC-1 Cells:

In human pancreatic cancer cells, CTB-GFP shows moderate accumulation at the cell membrane and scattered foci in the cytoplasm. PTD-GFP localizes similarly at the cell membrane but the signal is stronger in the cytoplasm than that of CTB-GFP. PG1-GFP shows very strong accumulation at the cell membrane but only scattered weak signals in the cytoplasm. RC101-GFP displays the strongest GFP levels both at the cell membrane and in the cytoplasm.

### HPDE Cells:

In human pancreatic ductal epithelial cells, CTB-fused GFP shows relatively strong, dense signals at the cell membrane and in the cytoplasm. PTD-GFP gives no clear signal at the cell membrane but has dense, moderate signals in the cytoplasm and the nucleus. PG1-GFP shows strong accumulation at the cell membrane but only scattered foci in the cytoplasm. RC101-GFP showed punctate GFP localization only at the cell membrane.

### HPDLSC Cells:

In human periodontal ligament stem cells, CTB-GFP shows relatively strong signals only at the cell membrane. PTD-GFP shows very weak accumulation at the cell membrane but has scattered, moderately bright GFP signals in the cytoplasm. PG1-GFP shows strong localization both at the cell membrane and in the cytoplasm. RC101-GFP fails to localize to both the cell membrane and the cytoplasm of these cells.

### MMSC Cells:

In maxillar mesenchymal stem cells, CTB-GFP shows relatively strong and dense localization to the cell membrane and scattered spots in the cytoplasm. PTD-GFP does not accumulate at the cell membrane but shows moderately bright foci in the cytoplasm. PG1-GFP shows extremely strong GFP signals at the cell membrane and scattered strong signals in the cytoplasm. RC101-GFP does not appear to localize anywhere in these cells.

### SSC-1 Cells:

In human head and neck squamous cell carcinoma cells, CTB-GFP shows weak GFP signals both at the cell membrane and in the cytoplasm. PTD-GFP displays relatively strong localization at the cell membrane and strong signals in the cytoplasm. PG1-GFP shows moderate accumulation at the cell membrane and scattered signals in the cytoplasm. RC101-GFP shows weak GFP localization at the cell membrane and a few scattered foci in the cytoplasm.

### RPE Cells:

In retinal pigment epithelium cells, CTB-GFP shows continuous (as opposed to punctate) localization at the cell membrane and relatively strong GFP foci in the cytoplasm. PTD-GFP displays relatively strong accumulation at the cell membrane and strong clustering in the cytoplasm. PG1-GFP shows strongest signals of any peptide in these cells at both the cell membrane and in cytoplasmic foci.

### GMSC Cells:

In gingiva-derived mesenchymal stromal cells, CTB-GFP localizes to both the cell membrane and the cytoplasm. PTD-GFP shows relatively strong GFP accumulation at the cell membrane and strong, clustered foci in the cytoplasm. PG1-GFP shows moderate accumulation of GFP at the cell membrane and strong, clustered signals in the cytoplasm.

### AGK Cells:

In adult gingival keratinocytes, CTB-GFP shows many relatively strong, dense signals in the cytoplasm. PTD-GFP also shows relatively strong signals in the cytoplasm, but there are comparatively fewer foci. PG1-GFP shows much stronger accumulation in the cytoplasm.

### OBC Cells:

In osteoblasts, CTB-GFP shows strong, punctate localization in the cytoplasm. PTD-GFP also strongly localizes to the cytoplasm. PG1-GFP only shows dense, continuous localization GFP at the cell membrane.

### DISCUSSION

Upon incubation with CTB-GFP, GFP signals were detected in all cell lines, and expression patterns in these cells were similar, consistent with the ubiquitous presence of GM1 receptors on membranes of these cells. Protein transduction domains (PTDs) also effectively facilitate the delivery of GFP into these cell lines and even transferred GFP into the nuclei of HPDE cells. These results confirm that PTDs can transfer foreign proteins directly without membrane receptors.

Protegrin 1 (PG1)-fused GFP shows strong localization to the cell membrane and the cytoplasm of most cell lines. There are three expression patterns:

1. Strong GFP signals at both the cell membrane and the cytoplasm (MMSC, RPE), showing that PG-1 can penetrate cell membranes.
2. Strong GFP signals at the cell membrane but weak or absent cytoplasmic signals (PANC-1, HPDE and OBC), which indicates that PG-1 cannot penetrate membranes of these cells.
3. Weak GFP signals at the cell membrane but strong signals in the cytoplasm (SCC-1, HPDLSC, GMSC and AGK). This pattern indicates that PG1 most easily and efficiently penetrates membranes of these cells and transports protein drugs efficiently into the cytoplasm. These results indicate that the efficiency of PG1-GFP penetration varies by cell type.
Protein drug delivery targets using fusion tags Fusions between CTB and therapeutic proteins facilitate effective oral delivery of therapeutic proteins for the induction of oral tolerance, delivery to serum, or even across blood-brain or retinal barriers. Foreign proteins can be delivered into living cells by fusing them with protein transduction domains (PTDs), which can penetrate cell membranes independently of specific receptors. PTDs can deliver biologically active proteins into cultured mammalian cells and in animal models in vivo and in vitro, giving PTD fusion proteins great potential for therapeutic drug delivery.

While antimicrobial peptides are known to kill microbes, human cell specific targeting is a novel and unexpected observation. Here we show that antimicrobial peptides can perform this dual function, making them ideal candidates to be used as peptide antibiotics as well as for effective delivery of other protein drugs in a cell specific fashion. A few examples of additional protein drugs are provided below. The GFP reporter protein amino acid sequence can be replaced with nucleic acids encoding the therapeutic proteins listed in table 4 which includes anti-inflammatory functional drugs, new treatments for pancreatitis, periodontal inflammation and periodontitis, pancreatic cancer and head and neck squamous cell carcinoma.

In the field of periodontal regeneration, several protein based therapeutics have been shown to have efficacy. Fusion of such proteins to the tags of the invention, following by administration of plant cells or plant parts expressing such fusion proteins should provide efficacy for the treatment of a variety of dental disorders.

TABLE 2
Candidate Growth Factors/Peptides for Periodontal Regeneration

| Growth | | |
|---|---|---|
| Factors/Peptides | Development Stage | Biologic Function |
| EMD | FDA approved | Enhances cell adhesion, |
| Enamel Matrix | | stimulated cell proliferation, |
| Derivatives | | angiogenesis, osteogenesis, |
| | | cementogenesis, and ECM |
| | | synthesis |
| P-15 | FDA approved | Enhances cell adhesion |
| 15 AA peptide | | |
| GTPGTQGIAGQRGVV | | |
| rhPDGF-BB | FDA approved | Increases chemotaxis of |
| Platelet derived growth | | inflammatory cells and MSC |
| factor | | progenitors, stimulates cell |
| | | proliferation, enhances |
| | | angiogenesis |
| rhFGF-2 | Phase II/III clinical trial | Stimulates fibroblast |
| Fibroblast growth | | proliferation and ECM |
| factor-2 | | synthesis, increases |
| | | chemotaxis, proliferation, |
| | | and differentiation of |
| | | endothelial cells |
| rhGDF-5 | Phase II clinical | Promotes cell proliferation, |
| Growth differentiation | | increases chemotaxis of |
| Factor 5 | | osteoblast progenitors, and |
| | | enhances osteoblast |
| | | differentiation |
| BMP-2 | Preclinical | Stimulates osteogenic |
| Bone morphogenic | | differentiation of MSCs |
| protein 2 | | |
| OP-I (BMP-7) | Preclinical | Increases mitogenesis and |
| Bone morphogenic | | differentiation of osteoblasts |
| protein 7 | | |
| BMP-6 | Preclinical | Enhances osteogenesis |
| Bone morphogenic | | |
| protein 6 | | |
| BMP-12 | Preclinical | Induces expression of |
| Bone morphogenic | | tendon- and ligament- |
| protein 12 | | specific genes, limited effect |
| | | on osteogenesis |
| BDNF | Preclinical | Stimulates osteogenesis and |
| Brain derived | | angiogenesis |
| neurotrophic protein 12 | | |
| PTH | Clinical | Bone anabolic effect |
| Parathyroid hormone | | |
| SOST scleorstin | Preclinical | Bone anabolic effect and |
| antibodies | | antiresorption |

Also see J. Periodontol. 86: S134-S152, 2015.

Table 3 provides a list of cells than can be selectively targeted via fusion with the different tags of the invention.

| | Protein | Cell Recruited |
|---|---|---|
| 1 | IGF-1 | Osteoblasts, Periodontal |
| | | Ligament (PDL) cells |
| 2 | FGF2 | Cementoblasts, Mesenchymal |
| | | Stem Cell, Osteoblasts, |
| | | Gingival Epithelium Cell, |
| | | Gingival fibroblasts, PDL cells |
| 3 | EMDs | Cementoblasts, Mesenchymal |
| | | Stem Cell, Osteoblasts, |
| | | Gingival Epithelium Cell, |
| | | Gingival fibroblasts, PDL cells |
| 4 | PDGF-BB | Cementoblasts, Menenchymal |
| | | Stem Cell, Osteoblasts, |
| | | Gingival fibroblasts, PDL cells |
| 5 | P-15 | Osteoblasts |
| 6 | BMPs | Mesenchymal Stem Cell, |
| | | OSteoblasts (>10 ng/mL, kill |
| | | PDL cells) |
| 7 | Del-1 | Neutrophil |
| 8 | GDF-5 | Mesenchymal Stem Cell, PDL |
| | | cells, Gingival fibroblasts |
| 9 | BDNF | PDL cells |
| 1 | PTH | Osteoblasts, PDL cells |

In addition, the approaches described herein can be employed to deliver drugs useful for the medical indications listed.

### Replacing Deficient Proteins for the Recited Disorders

### Endocrine Disorders (Hormone Deficiencies)

Insulin (analogues with faster action, longer duration)
Growth hormone (somatotropin)
Insulin like growth factor

### Hemostasis and Thrombosis (Post Translational Changes)

Blood clotting factors (VII, VIII, IX)
Anti-thrombin (surgical)
Protein C (venous thrombosis)
Tissue plasminogen activator (embolism, stroke)

### Replacing Deficient Enzymes

Enzyme deficiencies

### Beta-glucocerebrosidases

### Alpha-glucosidase

### Pulmonary/Gastrointestinal Disorders

Proteinase inhibitor (anti-trypsin deficiency)

### Lactase

### Lipase

### Amylase

### Protease

The therapeutic fusion proteins of the invention can also be used to augment existing pathways such as those involved in hematopoiesis and fertility. Thus fusion proteins including erythropoietin (anemia, chemotherapy), granulocyte colony stimulating factor, granulocyte macrophage stimulating factor, adenosine deaminases, human serum albumin (nephrotic syndrome), immunoglobulins, follicle stimulating hormone and chorionic gonadotropin can be produced. Different interferons, such as interferon alpha (hepatitis C, B antiviral) and interferon beta (multiple schlerosis) can be produced and administered as described herein.

Hormones such as parathyroid hormone and exenatide can also be produced as selectively targeted fusion proteins in accordance with the invention.

Bone morphogenic protein for treatment of spinal fusions, gonatropin releasing hormone for use in puberty, keratinocyte growth factor for oral mucositis associated with chemotherapy, and platelet derived growth factor for wound healing can be used.

Other protein based drugs can also be fused to the fusion peptides of the invention to improve targeted delivery. These include without limitation, botulinum toxin for dystonia and cosmetic uses, collagenases for severe burn treatment, DNase for cystic fibrosis, papain for burns and ulcers, asparaginase for leukemia, hirudin for coronary angioplasty and streptokinase for DVT or embolism.

Additional efficacious drugs can also be used in the fusion proteins of the invention and are listed in Table 4.

TABLE 4
The 20 top-selling biopharmaceutical products in 2013

| | | | Year | | Patent | Patent |
|---|---|---|---|---|---|---|
| | | Sales | first | | expiry | expiry |
| Ranking | Product | ($billions)^{a} | approved | Company | (EU) | (US) |
| 1 | Humira (adalimumab, anti- | 11.00 | 2002 | AbbVie & Eisai | 2018 | 2016 |
| | TNF) | | | | | |
| 2 | Enbrel (etanercept, anti- | 8.76 | 1998 | Amgen, Pfizer, | 2015 | 2028 |
| | TNF) | | | Takeda | | |
| | | | | Pharmaceuticals | | |
| 3 | Remicade (infliximab; anti- | 8.37 | 1998 | J&J, Merck & | 2015 | 2018 |
| | TNF) | | | Mitsubishi | | |
| | | | | Tanaba Pharma | | |
| 4 | Lantus (insulin glargine) | 7.95 | 2000 | Sanofi | 2014 | 2014 |
| 5 | Rituxan/Mab Thera | 7.91 | 1997 | Biogen-IDEC, | 2013 | 2016 |
| | (rituximab, anti CD20) | | | Roche | | |
| 6 | Avastin (bevacizumab; anti- | 6.97 | 2004 | Roche/Genentech | 2019 | 2017 |
| | VEGF) | | | | | |
| 7 | Herceptin (anti-HER2) | 6.91 | 1998 | Roche/Genentech | 2014 | 2019 |
| 8 | Neulasta (pegfilgrastim) | 4.39 | 2002 | Amgen | 2015 | 2014 |
| 9 | Lucentis (ranibizumab; anti- | 4.27 | 2006 | Roche/Genentech | 2016 | 2016 |
| | VEGF) | | | | | |
| 10 | Epogen/Procrit/Eprex/ESPO | 3.35 | 1989 | Amgen, J&J, | Expired | 2013 |
| | (epoetin alfa) | | | KHK | | |
| 11 | Novolog/Novorapid (insulin | 3.13 | 1999 | Novo | 2015 | 2015 |
| | aspart) | | | | | |
| 12 | Avonex (IFN-β-1a) | 3.00 | 1996 | Biogen Idec | 2015 | 2015 |
| 13 | Humalog mix 50:50 (insulin | 2.61 | 1996 | Lily | 2015 | 2014 |
| | lispro) | | | | | |
| 14 | Rebif (IFN-β-1a) | 2.59 | 1998 | Merck Serono | 2015 | 2013 |
| 15 | Aranesp/Nesp (darbepoetin | 2.42 | 2001 | Amgen KHK | 2016 | 2024 |
| | α) | | | | | |
| 16 | Advate/Recombinate | 2.37 | 1992 | Baxter | | |
| | (Octocog α) | | | | | |
| 17 | Levemir (insulin detemir) | 2.15 | 2004 | Novo | [Levemir] | 2014 |
| 18 | Actrapid/Novolin (insulin) | 2.02 | 1991 | Novo | 2017 | |
| 19 | Erbitux (cetuximab; anti- | 1.92 | 2004 | Bristol-Myers | 2014 | 2016 |
| | EGF) | | | Squibb, Merck | | |
| | | | | Serono | | |
| 20 | Eylea (aflibercept; anti- | 1.88 | 2011 | Regeneron, | 2020 | 2021 |
| | VEGF) | | | Bayer | | |

^{a}Financial data from LaMerie Business Intelligence. J&J, Johnson & Johnson

For more information on this table and other protein drugs, see Walsh, G. (2014) Biopharmaceutical benchmarks 2014. Nat. Biotechnol. 32, 992-1000.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for targeted delivery of therapeutic proteins to tissues or cells of interest in a subject to treat a disease or disorder in said subject comprising;
a) administering an effective amount of plant cells or remnants thereof comprising a plastid expressed nucleic acid encoding said therapeutic protein operably linked to a fusion peptide sequence, expression of said nucleic acid causing production of a therapeutic fusion protein in said plastid; and
b) improving or ameliorating symptoms associated with said disease or disorder caused by selective penetration of said therapeutic fusion protein into said tissues or cells of interest to the substantial exclusion of non target cells in vivo.

2. The method of claim 1, wherein said plant is selected from the group consisting of lettuce, tobacco, spinach, kale, cabbage, eggplant, carrot and tomato.

3. The method of claim 1, wherein said peptide is a PTD peptide.

4. The method of claim 1, wherein said peptide is a DC peptide.

5. The method of claim 1, wherein said peptide is an anti microbial peptide.

6. The method of claim 5, wherein said peptide is PG1 peptide or RC101 peptide.

7. The method of claim 1, wherein said peptide is a CTB peptide.

8. The method of claim 1, wherein said cells are immune cells.

9. The method of claim 1, wherein said cells are somatic cells.

10. The method of claim 1, wherein said cells are dendritic cells.

11. The method of any of claims 1-10, wherein said therapeutic fusion protein is for the treatment of an endocrine disorder and is selected from the group consisting of insulin, somatotropin, and insulin-like growth factor.

12. The method of any of claims 1-10, wherein said therapeutic fusion protein is for the maintenance of hemostasis or prevention of thrombosis and said protein is selected from the group consisting of a blood clotting factor, anti thrombin, protein C and tissue plasminogen activator.

13. The method of any of claims 1-10, wherein said subject has an enzyme deficiency and said therapeutic fusion protein is selected from the group consisting of beta gluco cerebrosidases, alpha-glucosidases, lactase, lipase, amylase, protease, and proteinase inhibitor.

14. The method of any of claims 1-10, wherein said therapeutic fusion protein augments existing treatment protocols and is selected from the group consisting of erythropoietin, granulocyte colony stimulating factor, granulocyte macrophage colony stimulating factor, follicle stimulating hormone, chorionic gonadotropin, alpha interferon, interferon B, PDGF, Keratinocyte growth factor and bone morphogenic protein.

15. The method of any one of claims 1-10, wherein said therapeutic protein is provided in Table 4.

16. A plant or plant cell comprising the therapeutic fusion protein of claim of any of the aforementioned claims.

17. The plant cells of claim 16 which have been freeze dried and are optionally in powdered form.

18. The plant cells of claim 16 which have been encapsulated.

19. The plant cells of claim 16, wherein said therapeutic fusion protein is stable at ambient temperature.
